(19) United States
(12) Patent Application Publication
(10) Pub. No.: US 2026/0311260 A1
(43) Pub. Date: Oct. 8, 2026

(54) Method And System For Estimating Impact Of Mutations On Fitness, Lifespan, And Fecundity

(71) Applicant: ILLUMINA, INC., San Diego, CA (US)

(72) Inventors: Hong Gao, Palo Alto, CA (US); Joshua Goodwin Jon McMaster-Schraiber, Berkeley, CA (US); Jacob Christopher Ulirsch, Cambridge, MA (US); Kai-How Farh, Hillsborough, CA (US)

(21) Appl. No.: 19/481,817

(22) PCT Filed: Jun. 20, 2024
(86) PCT No.: PCT/US2024/034840

§ 371 Date: Nov. 5, 2025

Related U.S. Application Data
(60) Provisional application No. 63/509,961, filed on Jun. 23, 2023.
(60) Provisional application No. 63/600,952, filed on Nov. 20, 2023.

(51) Int. Cl. G06Q 40/08; G06Q 40/06

(52) U.S. Cl. CPC G06Q 40/0821; G06Q 40/0631; G06Q 40/09

(57) ABSTRACT

Techniques are described for estimating selection coefficients for missense mutations and PTVs. In certain embodiments, such estimates may be based on forward-time population modeling and/or deep learning to facilitate estimation of individual-level selection burdens and/or to identify individuals with increased (or decreased) genetic risk of reduced fitness (including lifespan and/or fecundity metrics) relative to a threshold, such as a statistically mean or median fitness, lifespan, or fecundity based on a subject's demographic data.

### CROSS-REFERENCE TO RELATED APPLICATION

This application is a 371 national stage application of PCT/2024/034840, filed Jun. 20, 2024, which claims the benefit of priority of U.S. Provisional Application No. 63/600,952, filed Nov. 20, 2023, and the benefit of U.S. Provisional Application No. 63/509,961, filed Jun. 23, 2023, the contents of which are each incorporated by reference herein in their entireties for any purpose.

### FIELD OF THE TECHNOLOGY DISCLOSED

The technology disclosed relates to the calculation of quantitative metrics related to survivability, lifespan, or fitness. As discussed herein, the quantitative metrics are derived for an individual based on the selection coefficients estimated for possible mutations (e.g., missense mutations, protein truncating variants (PTVs), and so forth) within a genome as well as estimation of the individual's selection burden, which may be estimated using deep learning or other machine learning techniques.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

The genetic architecture of human complex traits is shaped by the history of natural selection. By understanding the selective pressures that shaped human genetic variation, insight can be gained into the genetic underpinnings of human disease.

One goal of genetics research is to understand how mutations in the genome of a species impact disease and lifespan within that species. To this end, in the human context thousands of genome-wide association studies (GWASs) have identified an extensive catalog of common variants associated with diseases in humans. However, GWAS variants are largely restricted to common variants with very modest effects on disease risk that are individually and even in aggregate too weak to guide clinical management. In contrast, clinical sequencing of patients with dominant Mendelian disorders has uncovered rare pathogenic variants with strong penetrance and high clinical actionability. Most of these pathogenic variants have not been previously reported, due to negative selection preventing individual variants from rising to higher allele frequencies. Thus, standard GWAS is not well-powered to detect them due to their relative infrequency. Instead, rare variant association tests typically aggregate variant across each gene, resulting in the discovery of individual genes underlying disease pathogenesis without providing clear evidence for the effects of the individual variants. Correspondingly, due to the lack of comprehensive visibility into the extent, nature, and mechanisms by which rare and/or pathogenic variants are present across a gene or across a genome, it is generally not feasible to assess the impact of such variants on metrics that may be of interest, such as fitness or lifespan. Further, downstream metrics and/or use cases that might benefit from such information, such as financial modeling, risk modeling, insurance modeling, demographic planning and so forth may instead have to utilize less accurate estimations of lifespan or fitness or may instead use generic or statistical approximations.

An alternative to these traditional association methods is to estimate the genome-wide extent of rare variant burden for each individual, typically referred to as selection burden. Studies have leveraged estimates of the selective effects of loss of function (LoF) mutations, including protein-truncating variants (PTV) and genic deletions, and demonstrated that an individual's LoF mutation burden is associated with reduced reproductive success and lifespan. However, these studies have typically excluded deleterious missense mutations, which are an order of magnitude more common in the human genome than LoF mutations.

With the preceding in mind, it may be understood that there are many components of fitness, including lifespan and fecundity. However, it has been difficult to disentangle those effects due to the difficulty of estimating fitness in an unbiased way in humans.

### BRIEF DESCRIPTION

A technical effect of the techniques described in greater detail below is the quantification of effects of one or a plurality of variants on a subject's lifespan, fitness, or fecundity. Due to the aggregation of variants across each gene, it is currently not feasible using currently available data to assess the impact of specific variants on reproductive success and lifespan. In particular, the most impactful genes on these metrics of interest may typically be underrepresented, i.e., rarely seen, in the general population, and thus underrepresented or absent in genome-wide studies typically used to characterize the prevalence of the different variants observed for individual genes. Correspondingly, the presently disclosed techniques provide technical benefits including, but not limited to, identification and quantification of infrequently observed alleles having statistically significant impacts on fitness, fecundity, and lifespan. Clinical technical benefits include, but are not limited to, enabling precision genetic healthcare solutions (e.g., targeted genetic interventions or modifications, design and/or delivery of customized nucleic acid or amino acid sequence-based therapies or treatments to affected individuals, design or customization of variant-specific biochemical or pharmaceutical therapies or treatments, and so forth) to be developed, personalized, and applied to impacted individuals. Further, non-clinical technical benefits may include generating accurate, quantitative estimates of fitness, lifespan, or fecundity (in contrast to population-based estimates or absence of data) for use in parameterizing financial models, such as insurance models, risk models, demographic models, investment or portfolio models, and so forth.

Systems, methods, and articles of manufacture are described for estimating a fitness, lifespan, and/or fecundity metric based on selection coefficients, such as may be derived from variant pathogenicity classifiers generated by a pathogenicity scoring system. The quantitative metrics are derived for an individual based on the selection coefficients estimated for possible mutations (e.g., missense mutations, protein truncating variants (PTVs), and so forth) within a genome as well as estimation of the individual's selection burden, which may be estimated using deep learning or other machine learning techniques. Such implementations may include or utilize non-transitory computer readable storage medium storing instructions executable by a processor to perform actions of the system and methodology described herein. One or more features of an implementation can be combined with the base implementation or other implementations, even if not explicitly listed or described. Further, implementations that are not mutually exclusive are taught to be combinable such that one or more features of an implementation can be combined with other implementations. This disclosure periodically may remind the user of these options. However, omission from some implementations of recitations that repeat these options should not be taken as limiting the potential combinations taught in the following sections. Instead, these recitations are hereby incorporated forward by reference into each of the following implementations.

This system implementation and other systems disclosed optionally include some or all of the features as discussed herein. The system can also include features described in connection with methods disclosed. In the interest of conciseness, alternative combinations of system features are not individually enumerated. Further, features applicable to systems, methods, and articles of manufacture are not repeated for each statutory class set of base features. The reader will understand how features identified can readily be combined with base features in other statutory classes.

In one aspect of the discussed subject matter, methodologies and systems are described that quantify a lifespan difference attributable to genetic mutations. In accordance with such embodiments the stored instructions are executed on one or more processors of a computer system. The instructions, when executed, quantify a lifespan duration or effect for an individual based on a plurality of mutations. The stored instructions, when executed cause the one or more processors to perform acts comprising: receiving as an input one or more mutations of interest determined to be present in the individual; calculating a total selection burden for the individual based on the one or more mutations of interest; quantifying an estimated change in lifespan for the individual based on the total selection burden derived for the mutations of interest and an expected lifespan for the individual absent the mutations of interest; and automatically generating and providing a plurality of clinical treatment options based on one or both of the mutations of interest for the individual or the estimated change in lifespan. Alternatively, in other embodiments the expected lifespan or change in expected lifespan may be provided as an input to a financial model, insurance model, actuarial model, risk model, demographic model, and so forth for estimation or determination of a model outcome or recommendation, such as a financial product recommendation, an insurance quote, an investment portfolio suggestion, and so forth

In a further aspect, methodologies and systems are described that quantify a lifespan or lifespan difference attributable to genetic mutations. In accordance with such embodiments the stored instructions are executed on one or more processors of a computer system. The instructions, when executed, quantify a lifespan metric for an individual based on a plurality of mutations. The stored instructions, when executed cause the one or more processors to perform acts comprising: estimating selection coefficients for a plurality of mutations determined to be present in the individual; estimating a selection burden for the individual based upon the estimated selection coefficients; estimating a lifespan or lifespan difference for the individual based on the selection burden for the individual; comparing the lifespan or lifespan difference to one or more thresholds; and based upon the comparison, determining that a clinical action is indicated; and based upon the determination that a clinical action is indicated and on the threshold comparison, determining a clinical action from among a plurality of clinical action for the individual.

In a further aspect, methodologies and systems are described that estimate survival based on mutation data. In accordance with such embodiments the stored instructions are executed on one or more processors of a computer system. The instructions, when executed, estimate a survival metric based on a plurality of mutations. The stored instructions, when executed cause the one or more processors to perform acts comprising: computing a baseline variance for censored survival time of a cohort; computing a variance incorporating selection burden; and determining a difference between the variance and the baseline variance, wherein the difference corresponds to the effect size of selection burden on predicted survival time.

In a further aspect, methodologies and systems are described that estimate a survival metric for an individual based on mutation data. In accordance with such embodiments the stored instructions are executed on one or more processors of a computer system. The instructions, when executed, estimate survival metrics based on a plurality of mutations. The stored instructions, when executed cause the one or more processors to perform acts comprising: accessing or acquiring gene panel data for the individual, wherein the gene panel data for the individual comprises mutation data for the individual for the genes associated with the gene panel; calculating a selection burden for the individual based on the genes associated with the gene panel; calculating a survival metric for the individual based on the selection burden; and displaying on a user interface of the computer system a plurality of selectable clinical treatment options for the individual and generated based on the survival metric.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts that simulated allele frequency spectra (solid bars) with optimal parameter combinations resemble the observed AFS of gnomAD synonymous variants (shaded bars). Missense variants are separated into three mutational classes due to different de novo mutation rates, including transition mutations at CpG sites (CpGTi), transition mutations at non-CpG sites (nonCpGTi), and transversion mutations (Tv).

FIG. 2 depicts a heatmap of test statistics of a goodness-of-fit test across various combinations of parameters. The optimal parameter combination occurs at number of generations=530 and the ratio between census and effective population size=30. The color or intensity of the heatmap shows the negative log Pearson's chi-squared statistic (corresponding to −2*log likelihood ratio).

FIG. 3 depicts a scatter plot showing the selection-depletion curve generated by incorporating each of 36 selection coefficients into the forward time simulation using the optimal parameter combination. The y-axis shows the fraction of variants depleted by selection compared to the variants under neutral evolution, denoted as “depletion” metric.

FIG. 4 depicts that the normalized ratios between observed and expected numbers of missense variants in merged variant databases (gnomAD, Topmed, and UKBB) decrease along the decile bins of PrimateAI-3D scores in each gene. The points on the right show the normalized ratios of observed and expected (OE) numbers of PTVs in each gene. The middle line shows the median OE ratios of missense variants across all the genes. The bottom and top lines represent the median ratios across high and low pLI genes, respectively.

FIG. 5 depicts that the mean selection coefficients of missense variants increase along the decile bins of PrimateAI-3D scores in each gene.

FIGS. 6A and 6B jointly depict that the depletion of missense variants increases with pathogenicity scores of various classifiers in genes BRCA1, PCSK9, BRAF and SCN2A. Missense variants of each gene were separated in 10 bins based on their pathogenicity scores of each classifier and the depletion metric shown on y-axis was computed for each bin. The x-axis represents the median percentiles of pathogenicity scores of each bin. Each color or line type represents one classifier, including SIFT, Polyphen2, CADD, REVEL, DEOGEN2, PrimateAI, and PrimateAI-3D. Blue diamond shapes represent the depletion metrics of PTVs in each gene.

FIG. 7 depicts that PrimateAI-3D scores achieved the highest Spearman correlation between missense OE ratios and variant pathogenicity across genes compared to other 19 variant pathogenicity classifiers.

FIG. 8 depicts the distribution of rare variant burden of samples from Europe, Asia, and Africa at each selection coefficient cutoff. The black dots represent the median number of rare missense/PTV variants per ethnicity.

FIG. 9 depicts the distribution of selection burdens of samples from Europe, Asia and Africa at different selection coefficient cutoffs. The black dots represent the median selection burden per ethnicity.

FIG. 10 depicts the impact of selection burden on human lifespan. Survival curves of probands in the top and bottom 5% of the selection burden distribution are illustrated.

FIG. 11 depicts a scatter plot showing that both parental and proband lifespan are significantly reduced with selection burdens of rare missense variants and PTVs. The y-axis shows the residuals of parental or proband lifespan after removing the effects of covariates.

FIGS. 12A, 12B, 12C jointly depict bar plots showing the per variant hazard ratios of different categories of variants in proband survival (FIG. 12A), paternal (FIG. 12B) and maternal (FIG. 12C) survival analyses. X-axis represents the increase in the risk of death by 100*(HR−1) % contributed per variant in each category listed on y-axis, which includes synonymous (Syn), missense (Mis), and PTV variants. Missense and PTV variants are further divided into groups according to their selection coefficients(s). The number of variants in each category per sample (n) and the p-value (P) of the survival analysis are reported to the right of each bar.

FIGS. 13A, 13B, 13C jointly depict horizontal bar plots showing the estimated number of years of parental (paternal (FIG. 13B) and maternal (FIG. 13C)) and proband (FIG. 13A) life expectancy lost per mutation stratified by selection coefficients of missense/PTVs after adjusting for the effects of covariates. The average number of variants per sample (n) and p-value of regression (P) were labeled for each category of variants.

FIGS. 14A and 14B jointly depict heatmaps showing the negative log p-value of selection burden in the regression analysis of parental lifespan (right) and proband lifespan (left). Genes were stratified into five bins according to LOEUF scores shown in y-axis and missense mutations within each gene were stratified by PrimateAI-3D quintile bins. Selection burdens were generated using only variants in each LOEUF/PrimateAI-3D bin. PTVs were treated as a separate group of variants with PrimateAI-3D score 1.0. Shading of the heatmaps represent −log 10 p-values of selection burden generated using rare missense variants/PTVs with MAF≤0.01% in the lifespan regressions.

FIGS. 15A, 15B, 15C jointly depict the impact of selection burden on proband and parental survival and lifespan stratified by LOEUF or pLI scores of genes and PrimateAI-3D scores of missense variants within genes. FIGS. 15A-15C depict heatmaps showing the negative log 10 p-values of selection burden in proband survival (FIG. 15C), paternal (FIG. 15A) and maternal (FIG. 15B) survival analyses. Genes were stratified into quintile bins according to LOEUF scores shown in y axis and missense mutations within each gene were stratified by PrimateAI-3D quintile bins. Selection burdens were generated using only variants in each LOEUF/PrimateAI-3D bin. PTVs were treated as a separate group of variants with PrimateAI-3D score 1.0. Colors of the heatmaps represent −log 10 p-values of selection burden generated using rare missense variants/PTVs with MAF≤0.01% in the survival analysis.

FIGS. 16A and 16B jointly depict heatmaps showing the negative log 10 p-values of selection burden in the regression analysis of proband lifespan (FIG. 16B) and parental lifespan (FIG. 16A). Genes were stratified into quintile bins according to pLI scores shown in y axis and missense mutations within each gene were stratified by PrimateAI-3D quintile bins. Selection burdens were generated using only variants in each pLI/PrimateAI-3D bin. Similarly, PTVs were treated as a separate group of variants with PrimateAI-3D score 1.0. Colors of the heatmaps represent −log 10 p-values of selection burden generated using rare missense variants/PTVs with MAF≤0.01% in the lifespan regressions.

FIG. 17 depicts a sample process flow by which a mutation component of lifespan may be derived, in accordance with one implementation of the technology disclosed;

FIG. 18 depicts a table illustrating the effects of rare variant burdens of top genes in parental and proband survival. Genes that are significant in at least one survival analysis after multiple testing correction are displayed in FIG. 18. The rare variant burden of each gene is defined as the number of PTVs plus the number of missense variants with the top 50% PrimateAI-3D scores in each gene. Genes are ordered as significant in father, mother, and proband survival. The last row shows the p-values of selection burden generated across all the genes in parental and proband survival analyses. P-values with stars exceed Bonferroni correction threshold.

FIG. 19 depicts that a multiallelic SNP dominates the effect of SRSF2 gene in reducing proband lifespan. The y-axis shows the distribution of proband lifespan for each group of SNP carriers.

FIG. 20 depicts selection burden impacts of common diseases. The contribution of selection burden to disease status is correlated with that disease's impact on lifespan. The x-axis shows the log-scaled hazard ratio of selection burden on the transition to disease status in the illness-death model. The y-axis shows the estimated years of life lost due to disease in the illness-death model.

FIG. 21 depicts that selection burden in known disease genes is specific to diseases linked to those genes. The x-axis shows gene panels, either from panelApp or from exome studies (see top bar to determine the provenance of a given panel). The y-axis shows a selection of diseases. Each cell shows the z-score by column of −log 10 (p-value) of selection burden in an illness-death model when only computed using genes in the panel on the x-axis and applied to the disease on the y-axis.

FIGS. 22A and 22B jointly depict scatter plots showing the selection burden of PanelApp gene sets with the strongest association signals (the most significant false discovery rates on the y-axis) with father (FIG. 22A) and mother (FIG. 22B) survival.

FIG. 23 depicts that the top PanelApp gene sets associated with father survival are consistent with those associated with mother survival. Colors or shading of the points represent different disease categories. “Develop” stands for developmental and neuromuscular disorders, “Cardio” for cardiovascular disorders, “Dysmorph” for dysmorphic and congenital abnormality syndromes, “Derm” for dermatological disorders, “Endo” for endocrine disorders, “Metab” for metabolic disorders, “RenalU” for renal and urinary tract disorders, and “Skeletal” for skeletal disorders.

FIG. 24 depicts a scatter plot that shows the fraction of variants transmitted from parents to offspring among the 877 trios identified in UKBB.

FIG. 25 depicts the fraction of variants transmitted from parents to unaffected offspring among 5,097 ASD trios decrease with selection coefficients of variants (p-value not significant).

FIG. 26 depicts simulations of variant transmission in trios with different sample sizes that show when sample size is insufficient, the power is limited to detect the reduction of allele transmission among deleterious variants. Different shading represents different simulated sample sizes. For each sample size, 10 data sets were sampled randomly from UKBB. For each dataset and each value of odds ratio of variant selective effects, 100 simulations were performed to compute the power of logistic regression analysis of allele transmission on variant selective effects.

FIG. 27 depicts a scatter plot showing that the number of offspring decreases with selection burden of males, irrespective of obtaining college degrees or not.

FIG. 28 depicts that when stratified by obtaining college degrees or not, the number of offspring decreases sharply in females with degrees. The y-axis shows the residuals of the number of offspring per sample after controlling for covariates.

FIG. 29 depicts a heatmap showing the probability that a variant with a true PAI percentile (x-axis) falls into the observed PAI percentile interval (y-axis) using SCN2A gene as an example. The sum of each row is normalized to one.

FIG. 30 depicts a sample process flow of steps in determining the effect of selection burden on survival time prediction, in accordance with one implementation of the technology disclosed;

FIG. 31 depicts a sample process flow of steps in determining a survival metric based on a gene panel, in accordance with one implementation of the technology disclosed.

FIG. 32 depicts a sample process flow of steps in determining clinical actions based on a fitness metric, in accordance with one implementation of the technology disclosed.

FIG. 33 depicts a sample process flow of steps in determining clinical actions based on a lifespan metric, in accordance with one implementation of the technology disclosed.

FIG. 34 depicts a sample process flow of steps in determining clinical actions based on a fecundity metric, in accordance with one implementation of the technology disclosed.

FIG. 35 depicts a pathogenicity score distribution, in accordance with one implementation of the technology disclosed.

FIG. 36 depicts, in tabular form, death ratios broken down by selection burden deciles of males and females, in accordance with one implementation of the technology disclosed.

FIG. 37 depicts, in tabular form, claim cost per policy for selection burden deciles, in accordance with one implementation of the technology disclosed.

FIG. 38 is a simplified block diagram of a computer system that can be used to implement the technology disclosed.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

As discussed herein, one goal of genetics research is to understand how mutations in a given genome may impact disease and lifespan within a species. To this end, within the human context, thousands of genome-wide association studies (GWASs) have identified an extensive catalog of common variants associated with diseases. However, GWAS variants are largely restricted to common variants with very modest effects on disease risk that are individually and even in aggregate too weak to guide clinical management. In contrast, clinical sequencing of patients with dominant Mendelian disorders has uncovered rare pathogenic variants with strong penetrance and high clinical actionability. Most of these pathogenic variants have not been previously reported, due to negative selection preventing individual variants from rising to higher allele frequencies; thus, standard GWAS is not well-powered to detect them. Instead, rare variant association tests typically aggregate variant across each gene, resulting in the discovery of individual genes underlying disease pathogenesis without providing clear evidence for the effects of the individual variants.

An alternative to these traditional association methods has been to estimate the genome-wide extent of rare variant burden for each individual, typically referred to as selection burden. Previous studies leveraged estimates of the selective effects of loss of function (LoF) mutations, including protein-truncating variants (PTV) and genic deletions, and demonstrated that an individual's LoF burden is associated with reduced reproductive success and lifespan. However, these studies have excluded deleterious missense mutations, which are an order of magnitude more common in the human genome than LoF mutations.

As may be appreciated, there are many components of fitness, including lifespan and fecundity. However, it has been difficult to disentangle those effects due to the difficulty of estimating fitness in an unbiased way in humans. With this in mind, and as discussed herein, techniques are described for inferring selection coefficients of missense and protein truncating variants in the human genome. Such techniques allow rare, deleterious mutations to be assessed for their impact in shortening human lifespan, reducing fecundity, and increasing developmental disorders.

As discussed herein, the selection coefficients of missense variants are based on depletion of variants among large human cohorts. Individual-level selection burden was estimated using UK biobank samples which were collected between 2010 and 2016. The impact of selection burden on human lifespan and reproductive success was observed using these approaches. It is expected that such knowledge pertaining to deleterious mutants and their impacts on fitness, fecundity, and lifespan may be used in clinical settings, such as in deriving prognoses for a subject, selecting and delivering treatments for a disease state, and/or pre-emptively taking preventative measures to address or mitigate genetic diseases that have yet to manifest, such as at an earlier age than such a disease might typically be observed to have a clinical consequence.

By way of further detail and discussion, techniques are described herein by which the strength of selection against every coding variant in the human genome may be estimated. In studies using these approaches, it was observed that most humans carry ~12 variants with a selection coefficient larger than 1%, indicating substantial deleterious variation in otherwise healthy individuals. By computing a per individual selection burden score, it was determined that individuals with more deleterious mutations have shorter lifespans, fewer offspring, and greater susceptibility to cancer and developmental disorders. It was further observed that the effect of selection burden on lifespan is partially mediated through common disease. That is, individuals carrying a higher burden of deleterious mutations are more prone to be diagnosed with common diseases that significantly impact lifespan. Thus, as discussed herein, leveraging evolutionary information about rare, deleterious mutations may be useful to further unlock the power of the genome in enhancing human health.

In certain embodiments discussed herein, techniques are described for estimating selection coefficients for all possible missense mutations and PTVs based on forward-time population modeling and deep learning to estimate these individual-level selection burdens and to identify individuals with increased (or decreased) genetic risk of reduced lifespan relative to a threshold lifespan, such as a statistically mean or median lifespan based on a subject's demographic data. As discussed herein, due to the expansion of the human population, average people carry deleterious mutation that have not been filtered by natural selection and that retain the potential to be highly pathogenic. In the context of understanding common diseases, GWAS data may be useful in identifying variants with weaker effects, while sequencing may be useful identifying variants with significant, large phenotypical effects. Advances in variant interpretation as discussed herein may facilitate quantifying the effects of such variants on lifespan, fecundity, and/or fitness.

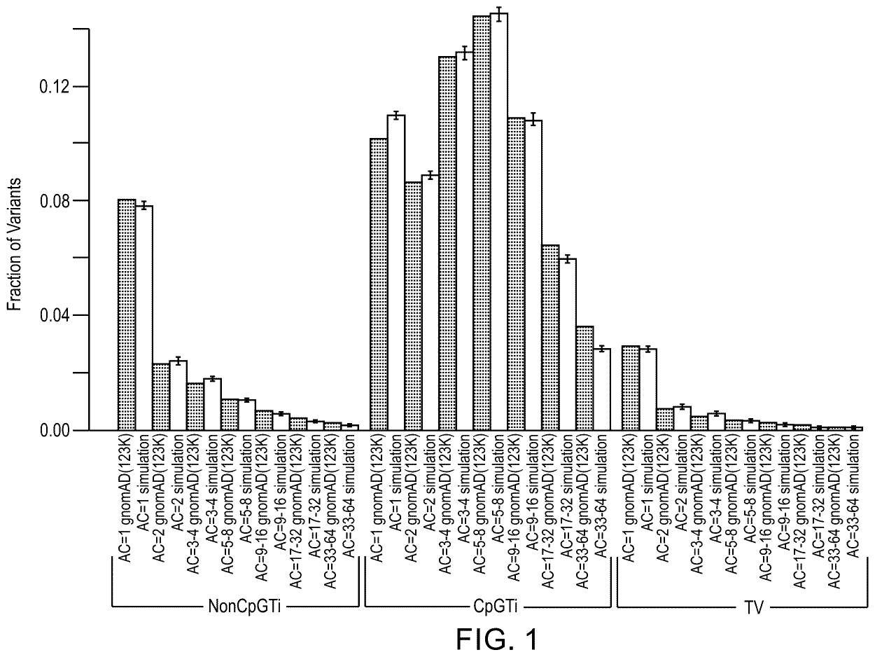

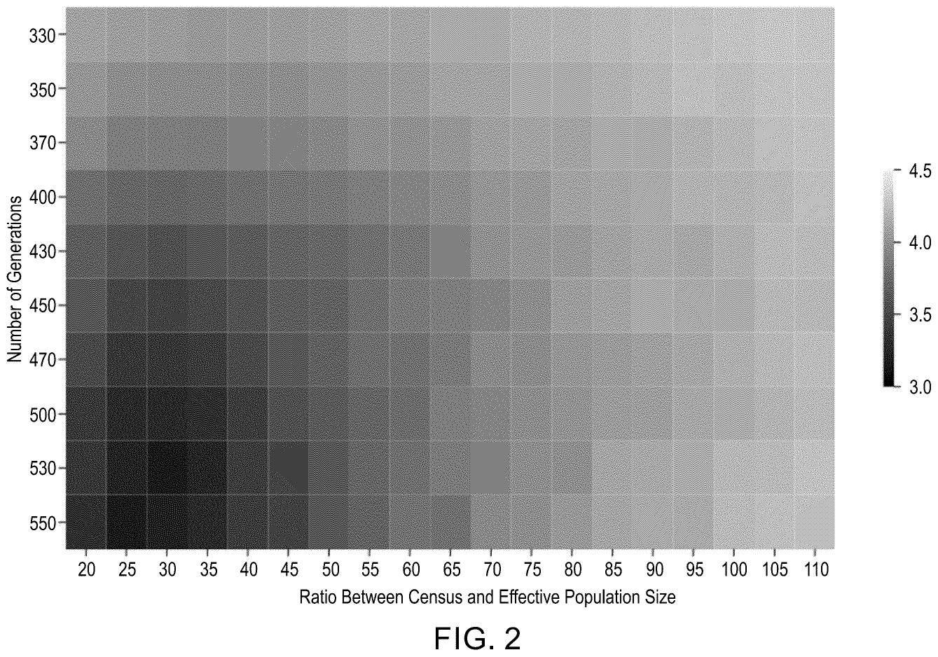

Estimating the fitness consequences of all possible human coding mutations—With the preceding in mind, studies and analyses were performed to assess the ability to identify the number and severity of deleterious mutations in a typical individual, which may be useful for providing precision genomic healthcare. By way of example, such precision genomic healthcare may include, but is not limited to, personal targeted genetic modification (such as via CRISPR techniques to modify or inactivate gene, promoter, or enhancer sequences), personalized nucleic acid or protein therapeutics, novel or designed pharmaceutical treatments based on a subject's unique biochemical profile or phenotype, and so forth. To quantify the severity of missense mutations in the human genome, in certain implementations a forward time population genetic modeling approach was employed to estimate the strength of selection against each potential variant. To do so, exome sequencing of 123,000 individuals from five major population groups and the known human de novo mutation rates in 192 trinucleotide contexts were leveraged. Parameters for population exponential growth modeling were first fit by matching the observed allele frequency spectrum for rare (AF<0.1%) synonymous mutations (FIG. 1). The present-day observed human allele frequency spectrum for synonymous mutations was found to best be explained by a very small initial effective population size (Ne=10,000) followed by exponential growth beginning 530 generations before present (FIG. 2), consistent with archaeological evidence for the invention of agriculture (e.g., ~10,000 BC, characterized as the beginning of Neolithic era).

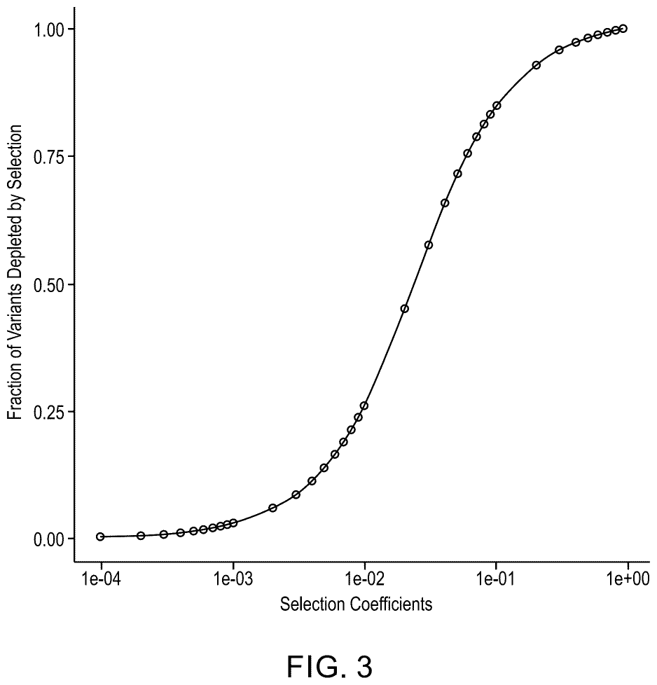

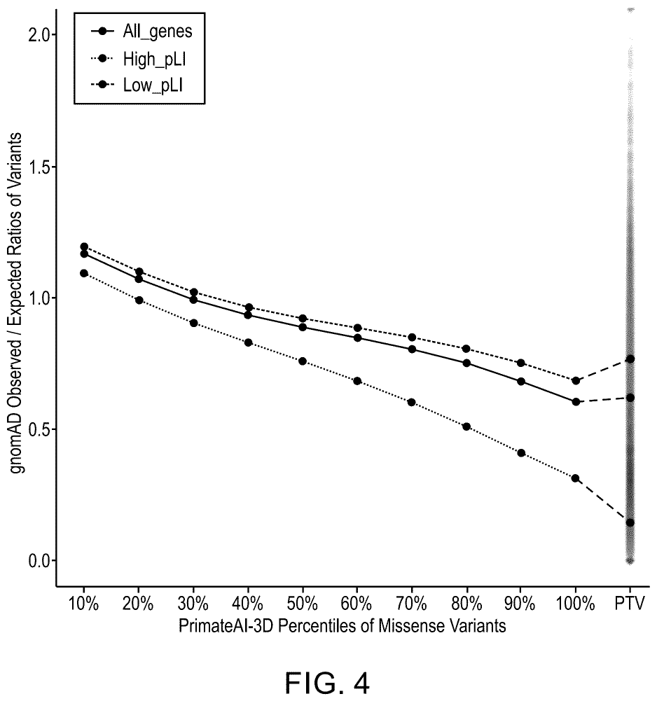

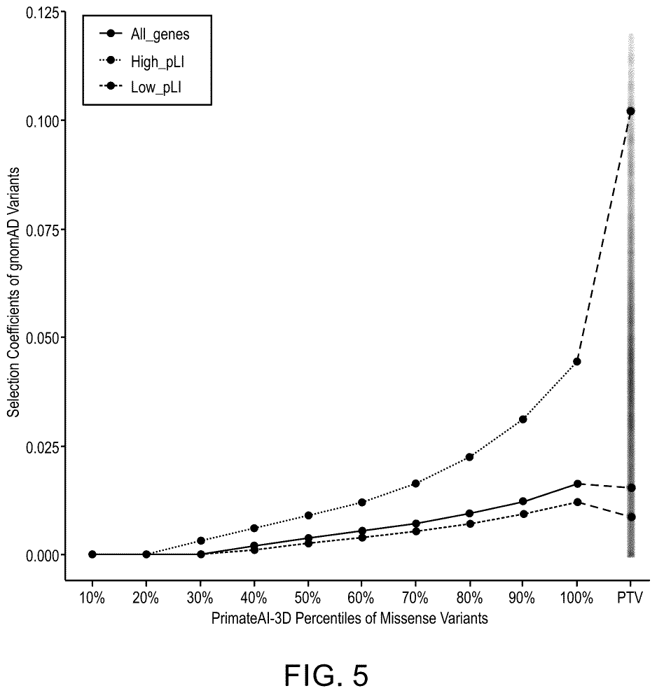

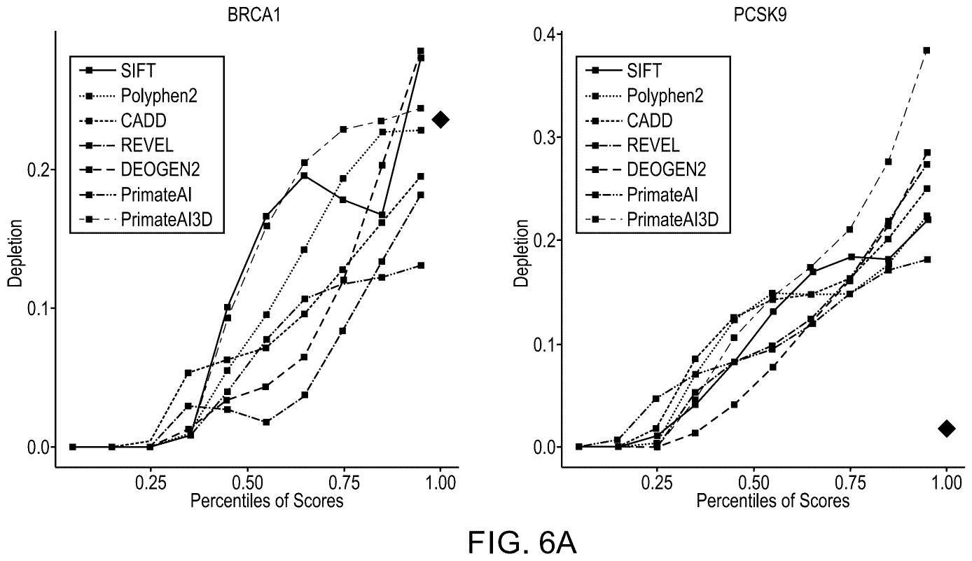

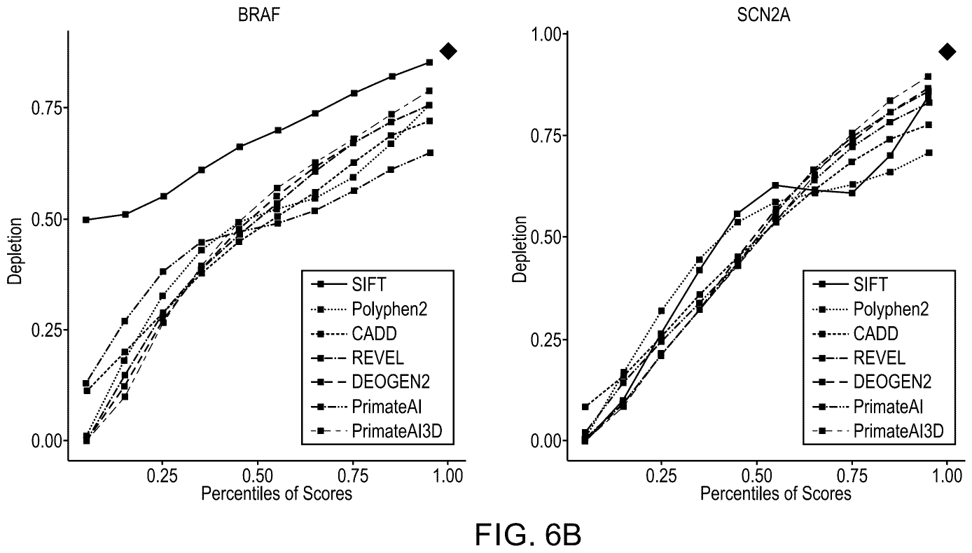

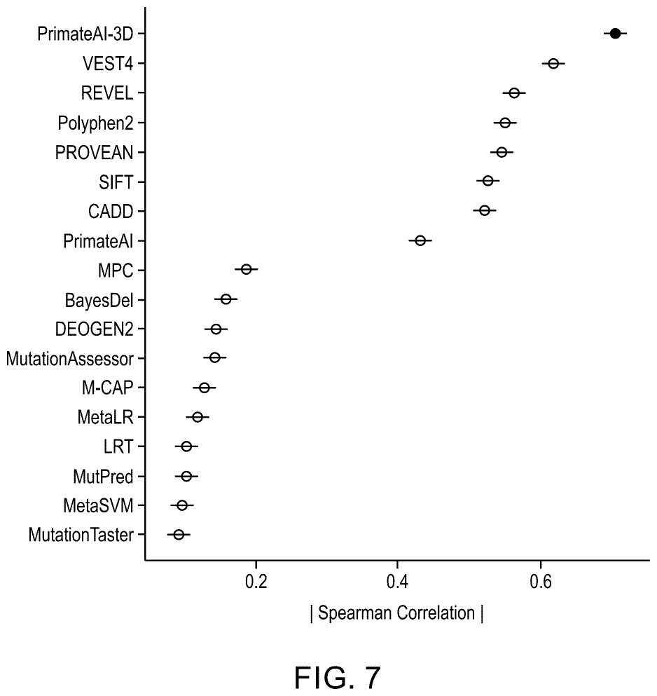

In these studies, to incorporate selection, simulation was performed across a grid of selection coefficients using the best fit demographic model and the depletion of selected variants relative to neutral variants computed for each selection coefficient (FIG. 3). For each human gene, quantification was performed for the depletion of observed PTVs compared to the expected number based on trinucleotide-context-dependent mutation rates and gene-specific synonymous variant counts (FIG. 4). Subsequently the gene-specific selection coefficient was derived via linear interpolation that best matched observed depletion on the simulated depletion-selection curve (FIG. 5). For missense mutations, pathogenicity bins were created by using gene-wise deciles of 20 different scores, including SIFT, PolyPhen2, CADD, REVEL, PrimateAI, and PrimateAI-3D, a 3-D convolutional neural network which leverages evolutionary information from 800+ individuals from 233 closely related primate species, to predict pathogenicity of every missense mutation in the human genome with accuracy. Although the various methods for estimating the pathogenicity of mutations varied in the functional relationship between pathogenicity percentile and variant depletion by natural selection, the normalized ratios of observed and expected (OE) numbers of missense variants per decile decreased nearly monotonically with the percentile score of variants within each gene for each classifier, with most highly pathogenic mutations close to those of protein-truncating mutations (FIG. 4, FIGS. 6A and 6B). For the purpose of the studies discussed herein, PrimateAI-3D was determined to outperform other pathogenicity predictors by achieving the highest rank correlation between missense OE ratios and variant pathogenicity percentile (FIG. 7). Correspondingly, PrimateAI-3D scores were adopted for use in performing the studies and a smoothing spline was fit to generate the expected depletion for each possible missense variant within a gene, given its prediction score, and derived a selection coefficient for every protein-coding variant in the human genome (FIG. 5).

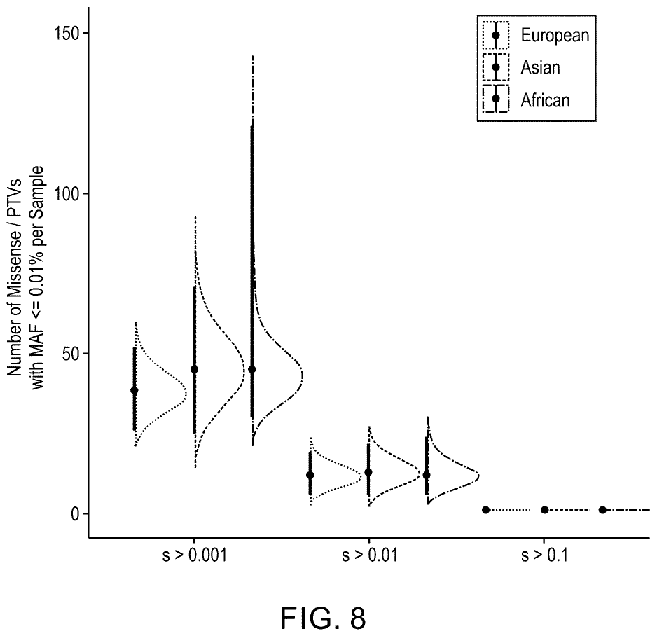

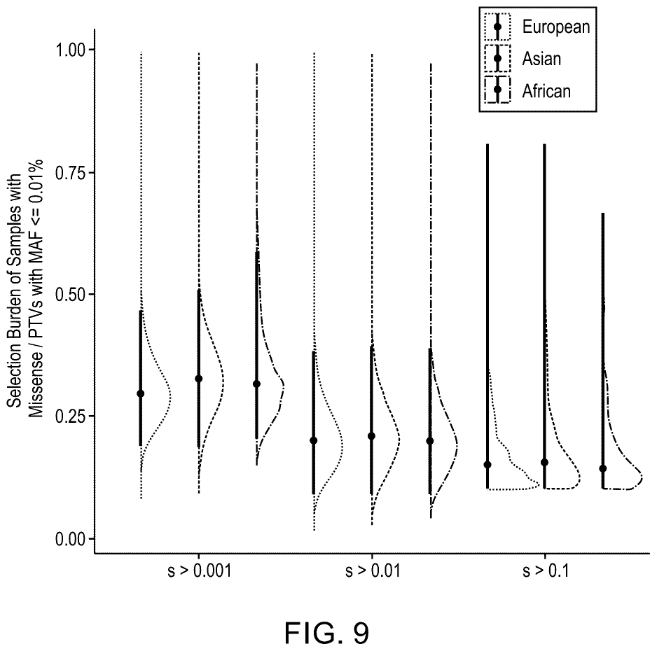

Rare, deleterious mutations and their association with lifespan—To determine the consequences of carrying rare deleterious variants, missense variants and PTVs were identified in UK Biobank (UKBB) individuals with birth years between 1934 and 1970 and a multiplicative mutation burden per sample computed using all mutations with genome Aggregation Database (gnomAD) allele frequency <0.01% and estimated selection coefficient >0.001, which is referred to herein as selection burden (FIG. 8). Most mutations carried by a person are under weak selection (s~0.001); however, it was observed that across self-identified ancestries, individuals carry roughly 12-13 mutations with s>0.01, and 1.1 mutations with s>0.1, indicating a substantial load of deleterious mutations in typical humans (FIG. 8). The distributions of individual deleterious mutation loads, as well as selection burden, were observed to be similar across different ethnic groups (e.g., European, Asian, and African) at each of the three selection cutoffs (FIG. 8 and FIG. 9).

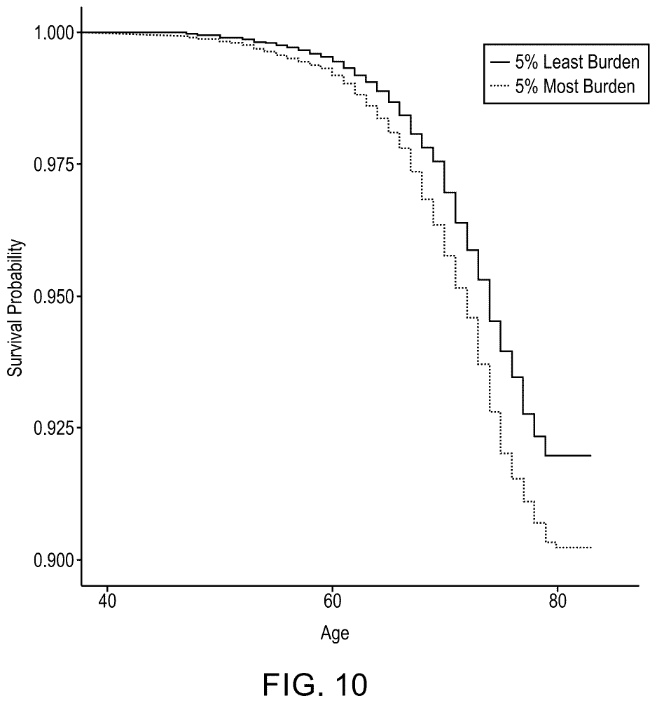

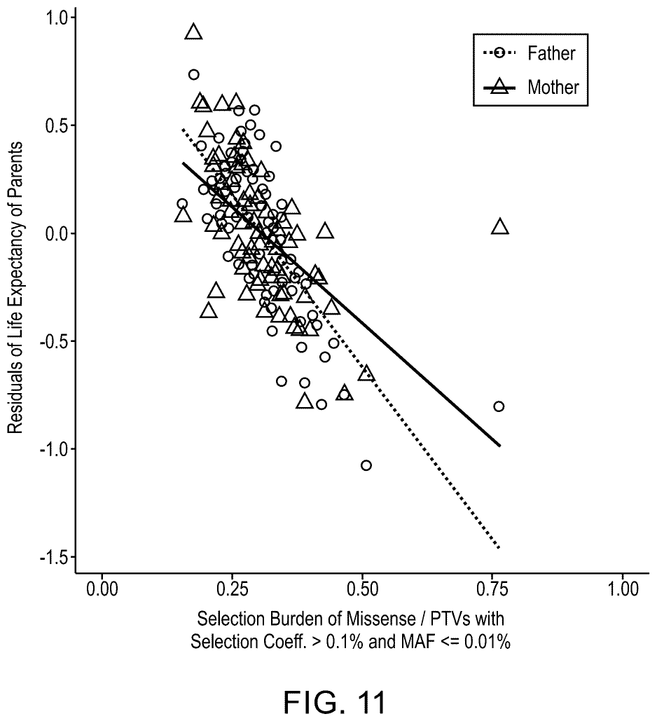

In a further aspect of the study, and based on the assumption that a major component of fitness is lifespan, the lifespans of 362,776 white British UKBB participants and their parents were analyzed to determine whether their selection burdens were associated with lifespan. As part of this analysis, Cox proportional hazards regression of proband lifespan was performed while adjusting for proband genetic principal components (PCs), proband gender, and age at recruitment. Significant effects were found for proband lifespan (hazard ratio (HR)=2.298, P=9.593e-15; FIG. 10). In particular, it was found that individuals with the 5% top deleterious burden in UKBB were expected to have 20% higher death probability and lose 0.80 years of life compared to the 5% least burdened individuals (FIG. 10). Proband results are shown in FIG. 10, while parental results are illustrated in FIG. 11. As may be appreciated, variants present in the proband are originally derived from the parents, so the association of lifespan was observed in parents as well.

The survival analysis was also applied to parental lifespan and significant effects were observed for both paternal (HR=1.320, P=4.694e-26) and maternal lifespan (HR=1.331, P=2.313e-22). To obtain interpretable quantification of the impact of selection burden on lifespan, a linear regression on lifespans was run following imputation using actuarial tables from the UK Office of National Statistics. It was observed that proband selection burden has a significant association with parental lifespan (for fathers' lifespan, effect size=−3.107, P=1.317e-23; for mothers' lifespan, effect size=−2.559, P=3.649e-18; FIG. 11) adjusting for proband covariates as described above. Moreover, including selection burden as a covariate increased the fraction of variance explained by 0.02%, 0.03%, and 0.2% for paternal, maternal, and proband lifespan, respectively.

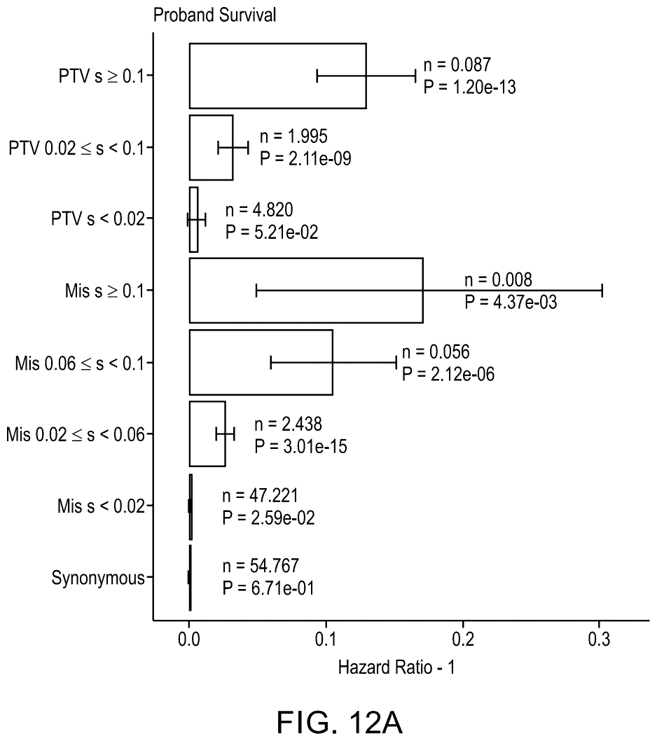

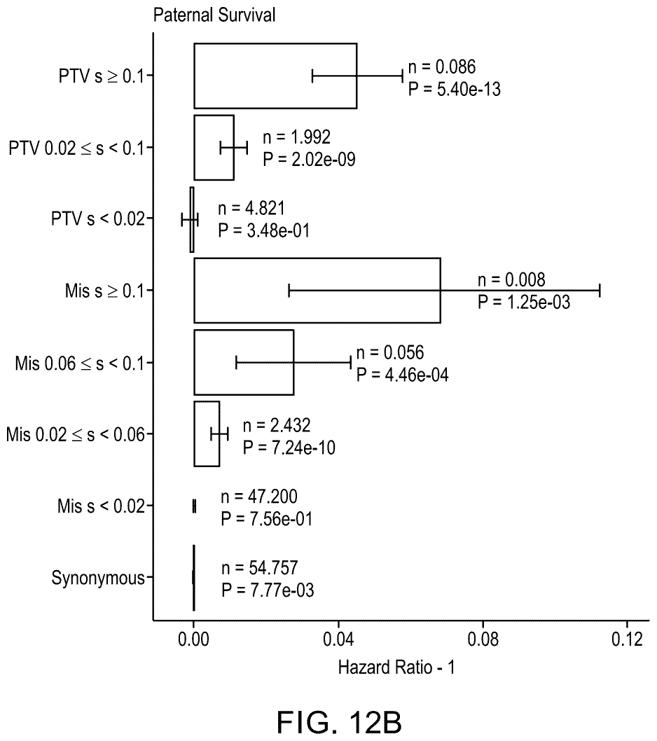

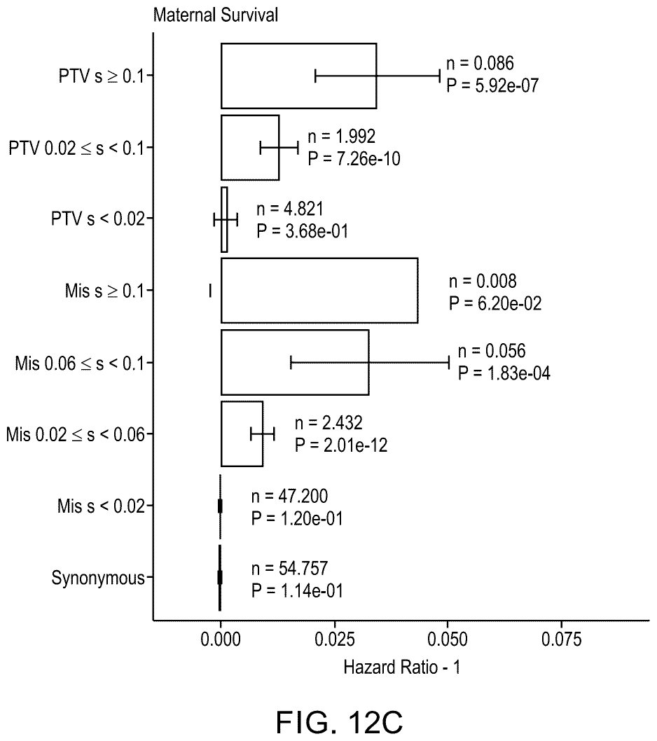

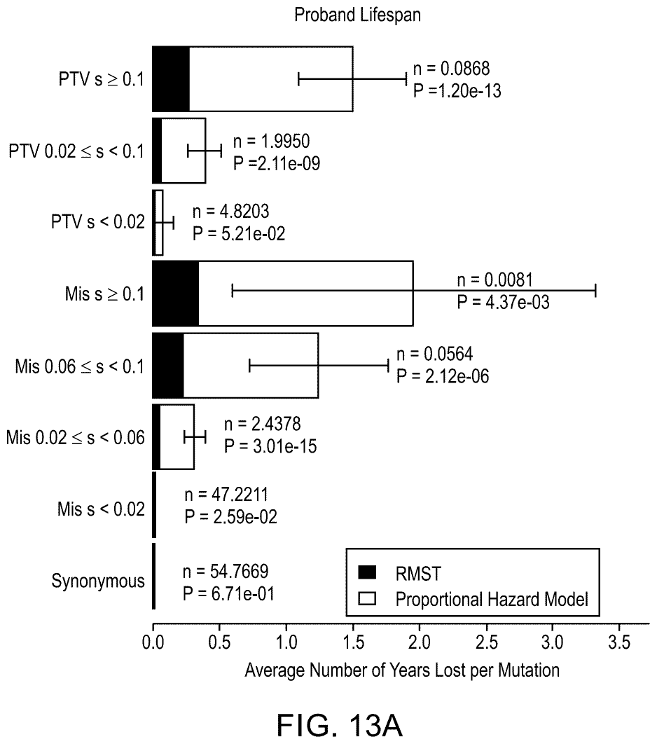

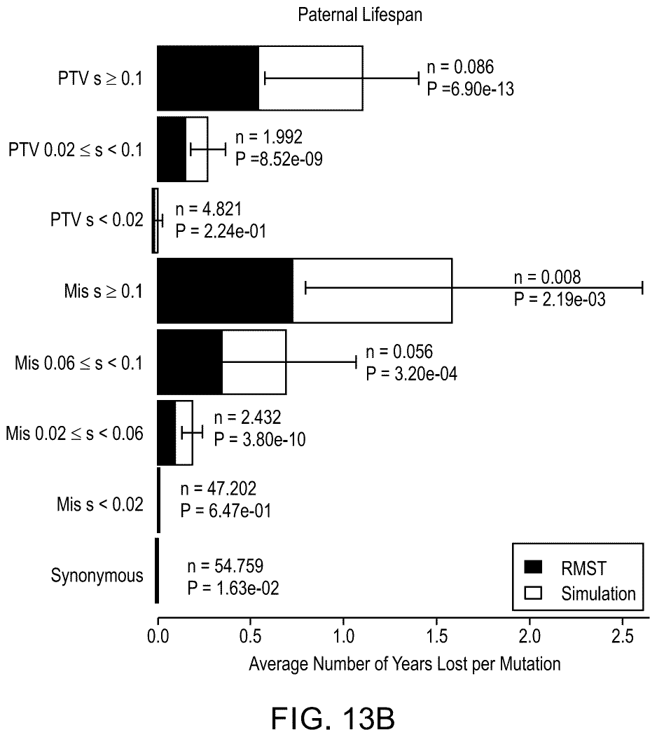

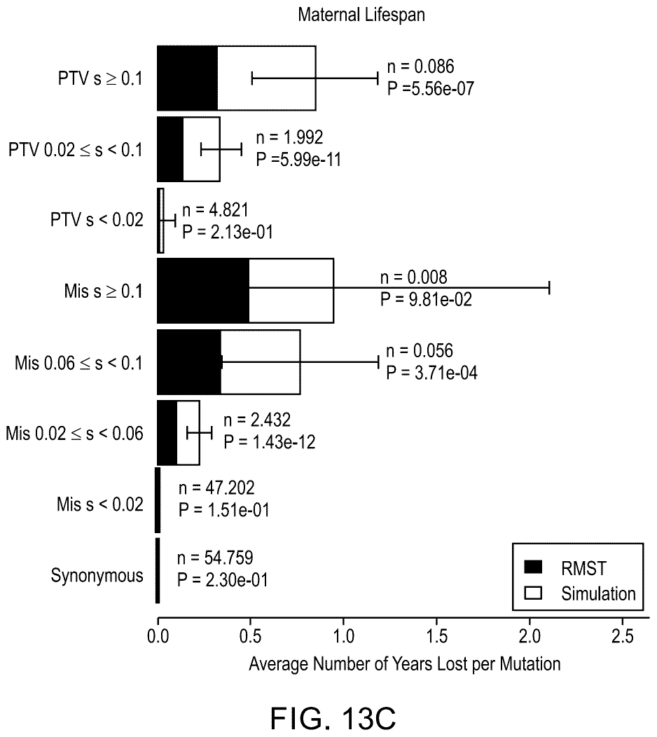

Which specific mutations and genes contributed most strongly to the effect on lifespan and survival was also examined. First, evaluations were performed on the impact of a single mutation on survival and lifespan separately for synonymous, missense and PTV variants. Per variant hazard ratios were obtained by applying proband and parental survival analysis while adjusting for covariates (FIGS. 12A, 12B, and 12C). On average, synonymous mutations, as well as missense and PTV mutations with small estimated selection coefficients (s<0.02), were found to have minimal effects on proband or parental survival. But highly deleterious mutations (s>0.1) increased the risk of proband death by 19.15%. To obtain interpretable per variant effect sizes, imputed lifespans were again used and either proband or parental lifespan were regressed on the number of mutations carried by an individual stratified by selection coefficients of missense/PTVs after adjusting for the effects of covariates (FIGS. 13A, 13B, and 13C). Similarly, synonymous mutations, as well as missense and PTV mutations with small estimated selection coefficients (s<0.02), did not appear to have statistically significant effects on lifespan. However, strongly deleterious mutations (s>0.1) were estimated to result in a decrease in proband lifespan of approximately 0.1 year each (FIGS. 13A, 13B, and 13C).

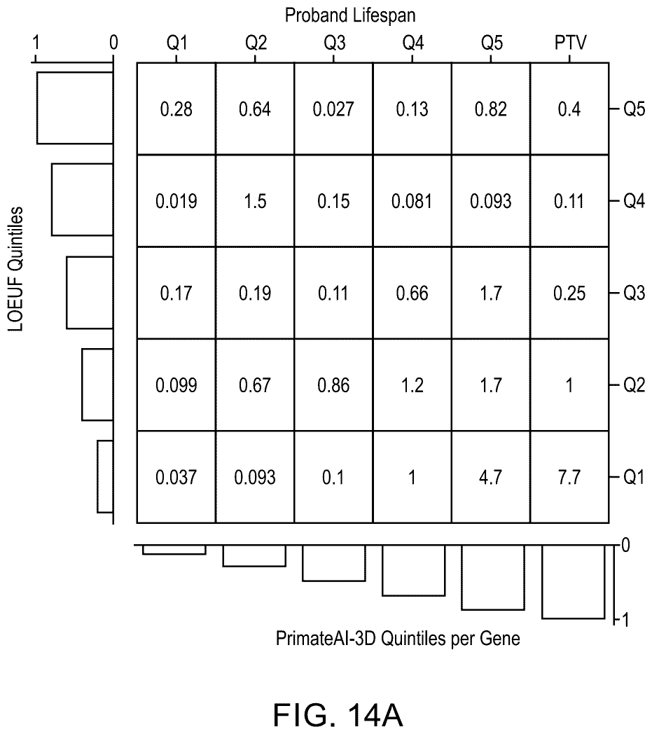

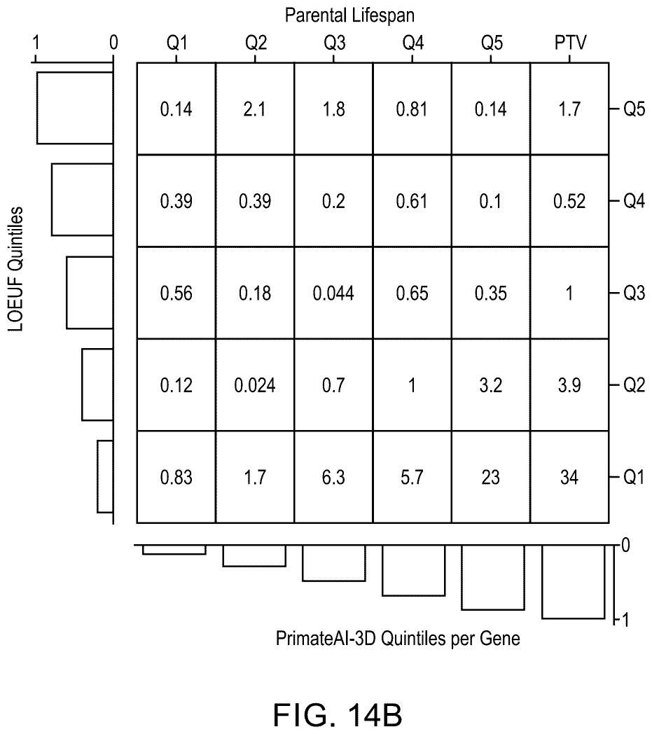

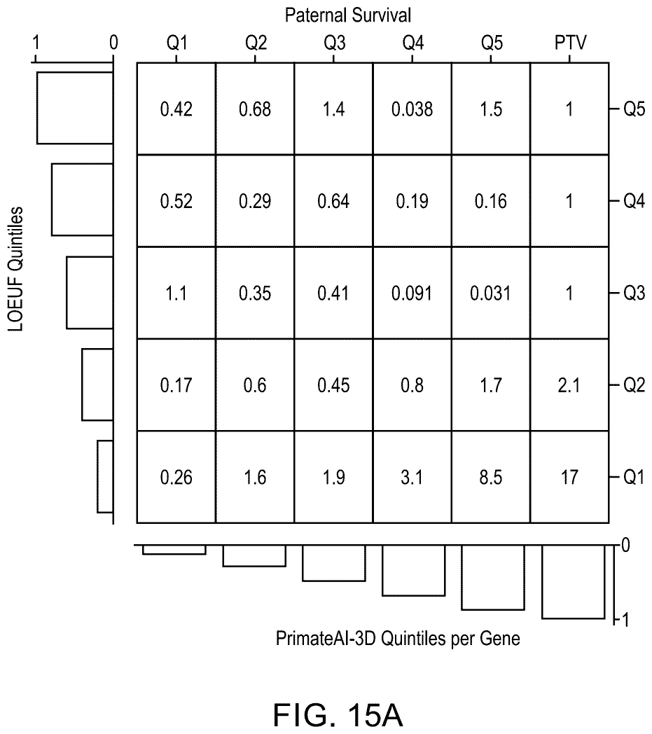

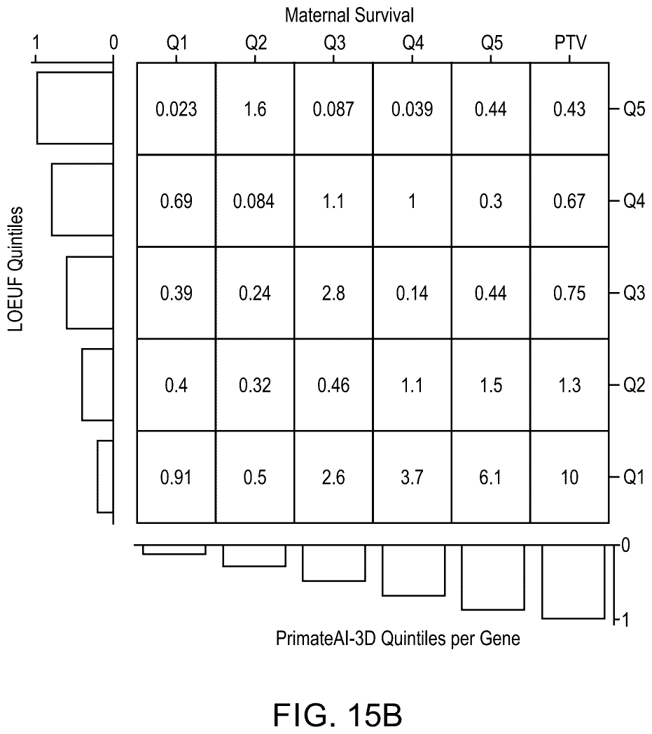

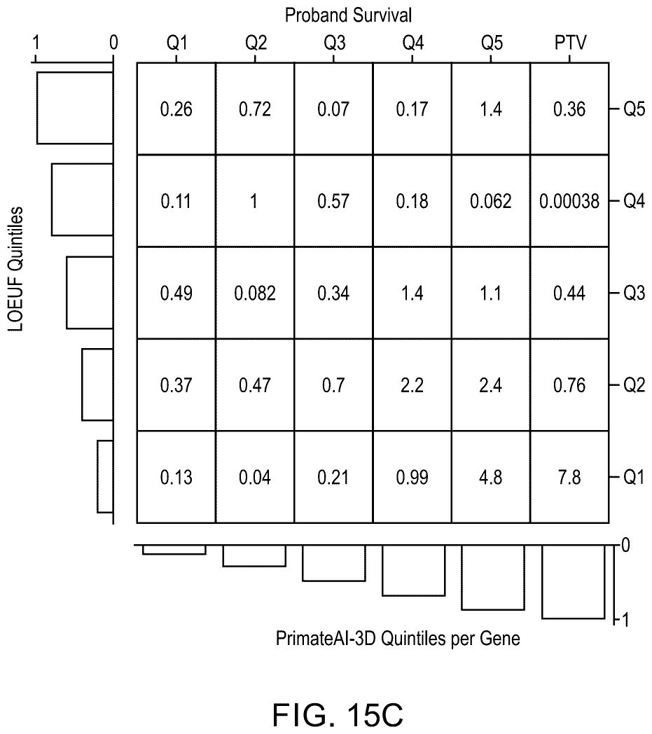

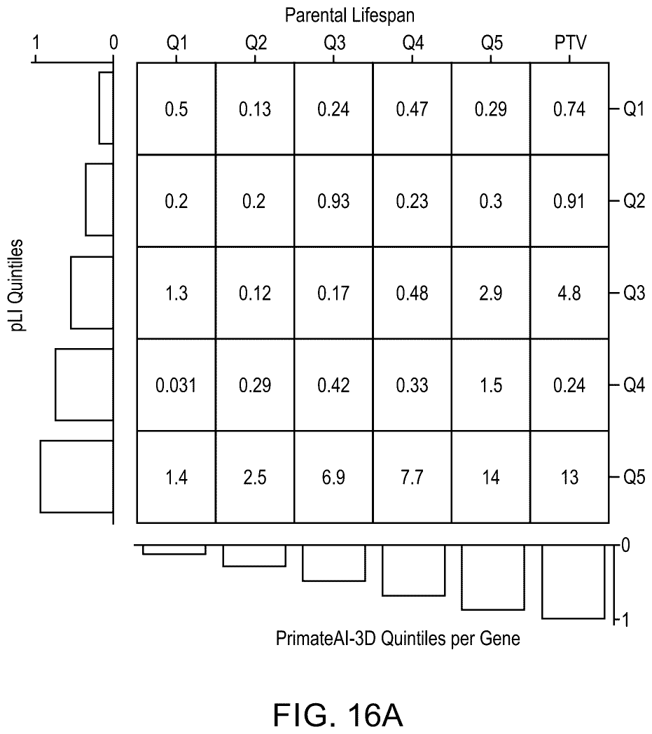

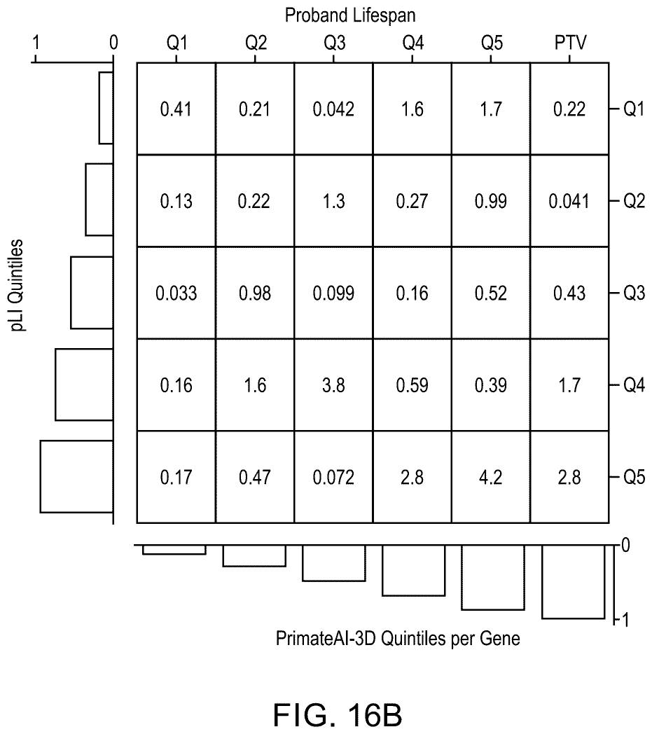

To investigate whether mutations in haploinsufficient genes were primarily responsible for lifespan shortening, genes were stratified into LOEUF score quintiles, which measure a gene's tolerance to loss of function mutations, then missense mutations within each gene were stratified by PrimateAI-3D quintile. The imputed lifespan regression analysis and survival analysis were repeated while computing selection burden restricting to only variants in each LOEUF/PrimateAI-3D bin, and it was determined which bins had a significant association with individual or parental lifespan (FIGS. 14A and 14B and FIGS. 15A, 15B, and 15C). It was found that strongly deleterious mutations (i.e., high PrimateAI-3D scores) in the most constrained genes (i.e., low LOEUF) made the strongest contributions to lifespan, reaffirming the importance of disruptive mutations in constrained genes in human lifespan. Interestingly, even mutations with moderate effects in genes under strong negative selection were observed to potentially have substantial effects on fitness, which was also observed when stratifying genes by pLI scores (FIGS. 16A and 16B). Notably, this identified a subset of genes where the most pathogenic missense mutations exceed the pathogenicity of protein truncating variants. Among this subset were well-known disease genes where the disease mechanism is known to be toxic gain-of-function or dominant negative effects.

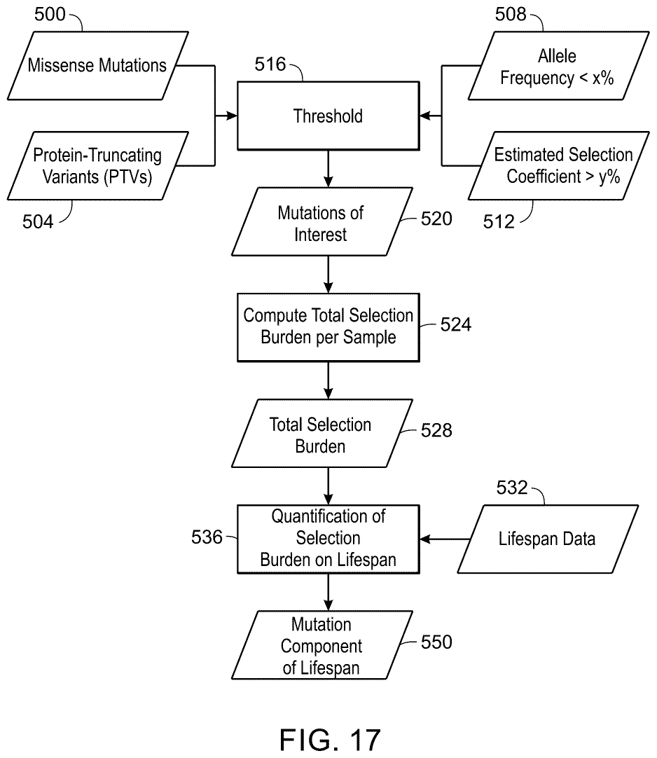

With the preceding in mind, and turning to FIG. 17, a process flow of steps, suitable for being performed by a processor-based system (e.g., desktop or notebook computer, workstation, or specialized sequencing device) are depicted. Such steps may be performed as part of a treatment or examination of a subject or patient or as part of a study protocol. Intermediate or final outputs or results of such steps may be provided as part of a report or diagnosis which may be stored on a processor-based system, displayed for review by such a system (such as on a user interface of such a system), printed for storage or review, or may be used as part of a decision making process based on the analysis, such as by displaying one or more selectable treatment options for the subject or by automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

With this in mind, and turning to FIG. 17, aspects of the above description are illustrated as steps of a process that may be used for quantifying a mutation component of a subject's potential lifespan for diagnosis or counseling of the subject or for other therapeutic or research uses. In the depicted example, a variety of initial data such as, but not limited to, sequence data identified as or characterized as known missense mutations 500, protein truncating variants (PTVs) 504 along with the corresponding allele frequencies 508 and estimated selection coefficients 512 for these mutations as well as all possible mutations in a human genome are processed against one or more thresholds (step 516) or other cutoff criteria to identify mutations of interest 520 for use in assessing a mutation component of lifespan 550. By way of example, cutoff criteria or thresholds employed may, in the case of missense mutations, correspond to the use of pathogenicity bins employing gene-wise deciles of one or more pathogenicity scores derived for each missense mutation using one or more scoring systems (e.g., SIFT, PolyPhen2, CADD, REVEL, PrimateAI, PrimateAI-3D, and so forth). Similarly, for PTVs, a threshold or cutoff may be based on a depletion metric or selection strength quantified based on the depletion of observed PTVs relative to an expected number based on trinucleotide-context dependent mutation rates and gene-specific synonymous variant counts.

Based on the mutations of interest 520 so identified, a total selection burden 528 may be computed (step 524) for a given sample obtained for a subject of patient. Estimated lifespan data 532 for the subject or patient, such as actuarial data or other lifespan data based on demographic and/or medical history may be used to quantify (step 536) a positive or negative component (e.g., a mutation component 550) of an estimated lifespan of the subject or patient. By way of example, the estimated mutation component 550 of lifespan may comprise a value in days, weeks, month, or years, that a subject's lifespan is expected to be increased or decreased based on their respective total selection burden 528 assessed for the mutations of interest 520. Such data may be displayed for review on a user interface of a processor-based system, and/or or may be used as part of a decision-making process, such as for identifying one or more treatment options, automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

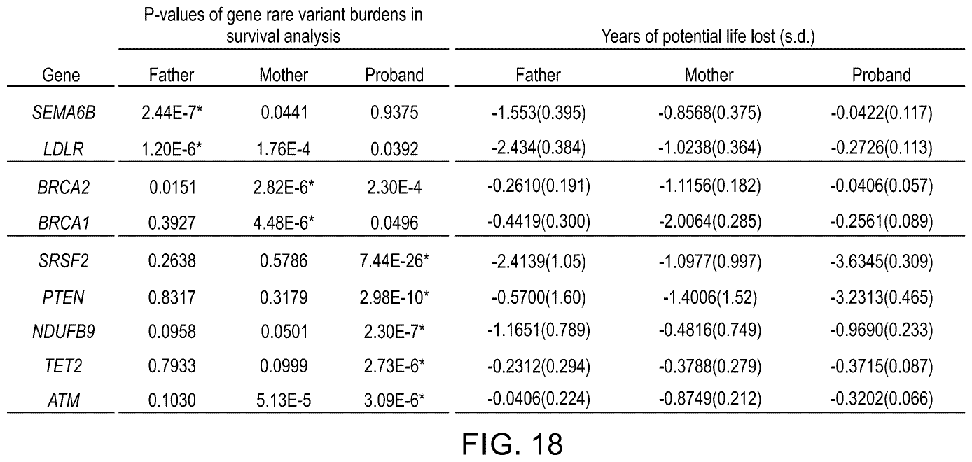

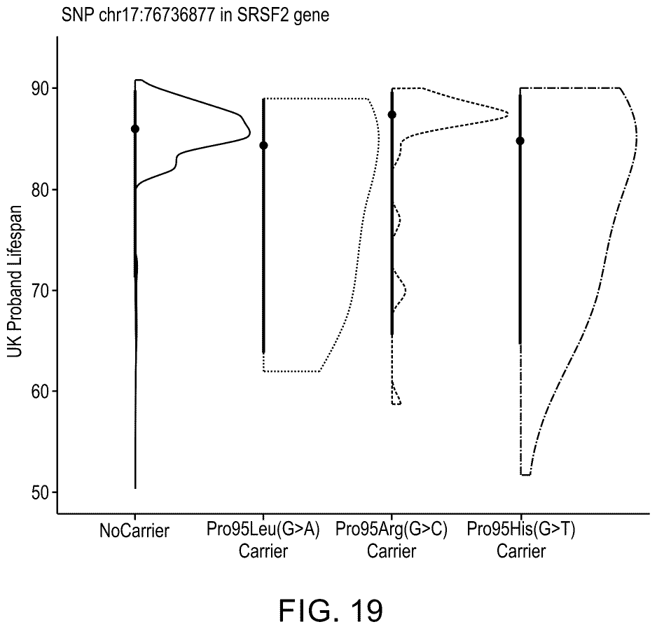

Genes associated with lifespan—In further aspects of the study, to identify specific genes that are strongly associate with lifespan, gene-burden survival analyses were conducted to search for individual loci that are associated with lifespan. When restricting to PTVs, BRCA1, BRCA2, ATM, and TET2 were identified as influencing lifespan either through a known role in cancer or due to clonal hematopoiesis. When rare missense variants from the top 50% of PrimateAI-3D scores in each gene were included in the analysis, 5 additional novel gene associations with lifespan were identified (FIG. 18). The strongest signals were observed for SRSF2 (HR=4.899, P=7.444E-26), PTEN (HR=6.175, P=2.976E-10) and NDUFB9 (HR=2.616, P=2.302E-07) with proband survival. SRSF2 is the most frequently mutated spliceosome gene in chronic myelomonocytic leukemia, and each deleterious mutation in this gene results in an average of 3.6 years of lost lifespan. The multiallelic SNP at chr17:76736877 dominates this effect and is a recurrent mutation during clonal proliferation (FIG. 19). Similarly, each mutation of PTEN, a tumor suppressor gene, was observed to lead to loss of 3.2 years in proband lifespan. LDLR (HR=1.179, P=1.20E-06) and SEMA6B (HR=1.185, P=2.44E-07) were significant when analyzing paternal survival, while no additional genes were significant when looking at maternal survival. LDLR is a well-known gene causal for familial hypercholesterolemia and early-onset cardiovascular disease, whereas SEMA6B is associated with epilepsy disorders. When these top nine genes were excluded from selection burden, the fraction of variance explained by recomputed burden was reduced by 2.5% for paternal survival time, 3.0% for maternal survival, and 7.0% for proband survival.

The impact of deleterious mutations on human lifespan as mediated by common disease—With respect to the impact of deleterious mutations on lifespan where the impact is mediated by disease, certain observations were made. Single genes, though statistically significant, explain very little of the total selection burden on lifespan. Thus, it was analyzed whether deleterious mutations may reduce lifespan via their accumulated effects on complex disease. As part of this study, illness-death models were applied to ~600 ICD10 codes with prevalence above 0.1% in UKBB. In these models, individuals begin in a “healthy” state at birth and then progress either directly into a dead state or proceed first through a disease state prior to death, which requires knowledge of the age of onset for the specific disease. Thus, the effect of selection burden on death was separated from the portion that is mediated via disease. For each disease, an illness-death causal mediation model (implemented in the R package mstate) was used to infer a Cox proportional hazards model of effect of burden for mutations with s>0.01 on transitions between each state while controlling for genetic PCs, sex, and age at recruitment. For late onset diseases, the observed years lost might be ten-fold, while for early onset diseases the years lost may need no adjustment.

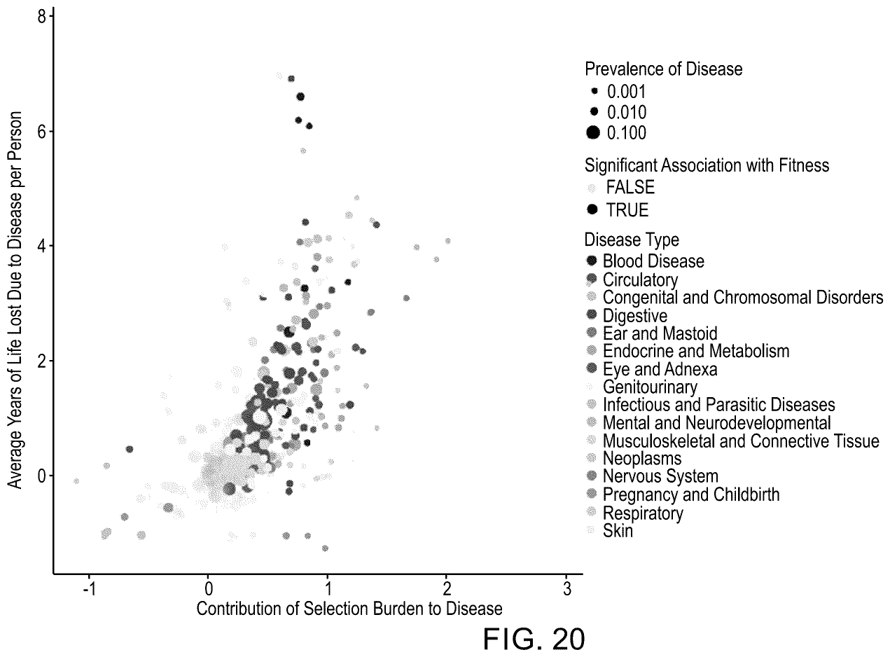

It was found that selection burden increases the rate of transition into the disease state for 241 diseases at a false discovery rate (FDR) of 5%, consistent with selection burden contributing to common disease. For example, it was found that selection burden significantly increases the rate of diagnosis with diabetes (HR=2.51, p=1.83E-42), obesity (HR=2.09, P=1.29E-31), and acute renal failure (HR=2.6, P=3.51E-21), indicating a substantial impact on common disorders known to impact lifespan. In contrast, selection burden only increased the rate of transition from disease to death for 35 diseases at FDR of 5%. The years of expected life lost per person to each disease in UKBB was then computed and a positive correlation found between years lost and the effect of selection burden on disease (FIG. 20, Spearman r=0.568, P=0) suggesting that the effect of deleterious mutations on lifespan is partially mediated by deleterious mutations increasing the rate at which individuals develop common diseases.

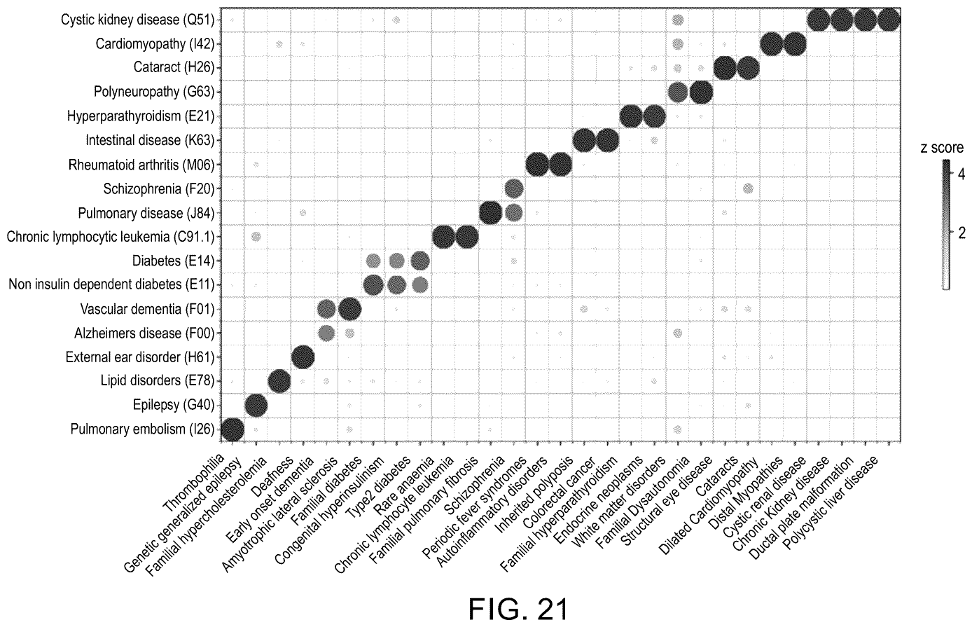

If the selection burden contributes to complex diseases, then the associations between selection burden and these diseases should remain when computing selection burdens restricted to genes that are known to be associated with each disease. Conversely, the effect of selection burden on other diseases should be diminished when computed using genes not known to be associated with those diseases. As part of this study, gene sets were compiled that had been associated with a disease either through exome rare variant association or via expert curation in PanelApp. Highly sensitive and specific effects of selection burden were observed when restricted to gene sets (FIG. 21). For example, selection burden restricted to genes that are associated with diabetes either from exome association studies or PanelApp was observed to specifically increase the rate of proband diagnosis with diabetes, while not impacting the rate of diagnosis of other diseases.

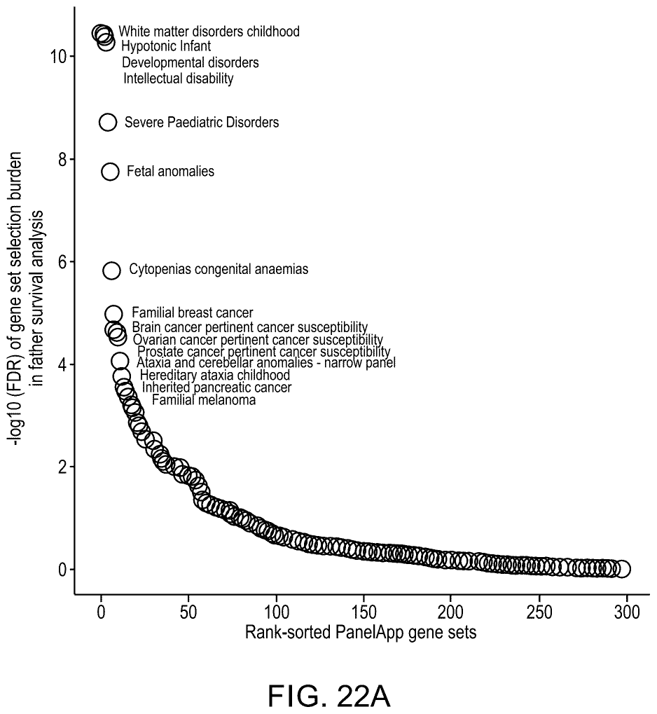

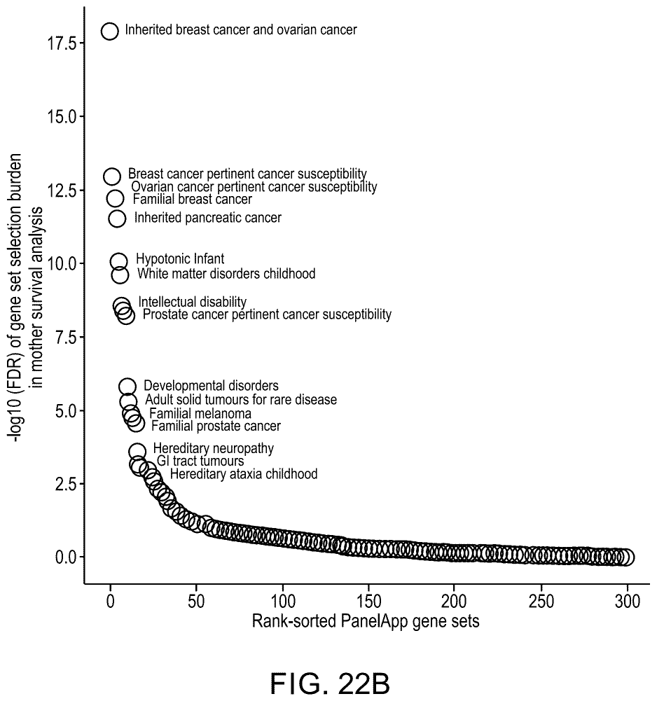

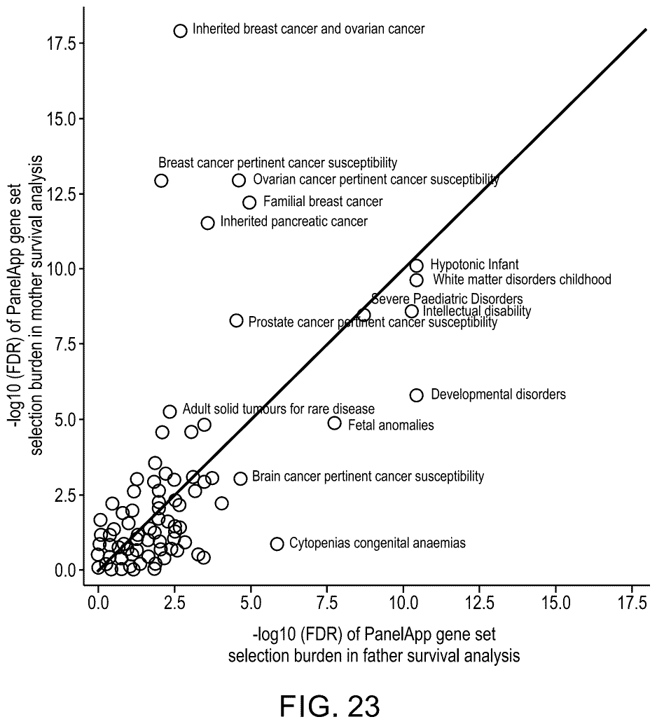

However, looking only at diseases that are common in the UKBB population substantially reduces the set of pathologies available to be examined. Thus, gene-set burden survival analyses were conducted using the 315 PanelApp curated gene panels for both parental and proband lifespans. Gene sets related to developmental disorders are among the strongest signals associated with fathers' survival, followed by cancer gene panels and gene sets related to cardiovascular disease, whereas breast and ovarian cancer gene panels top the list for mothers' survival (FIGS. 22A and 22B). Generally, the significance of gene panels is consistent between fathers' and mothers' survival (FIG. 23), although there is signal for sex specific effects, such as breast and ovarian cancer primarily impacting women and cardiovascular disease primarily impacting men. None of the PanelApp gene sets show significant association with proband survival after applying multiple testing correction, likely due to low power as 96% of probands were alive.

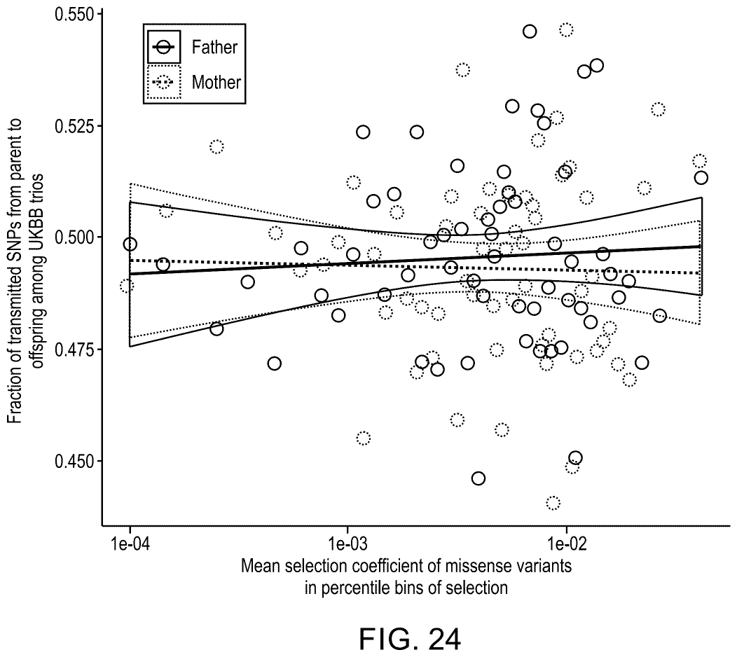

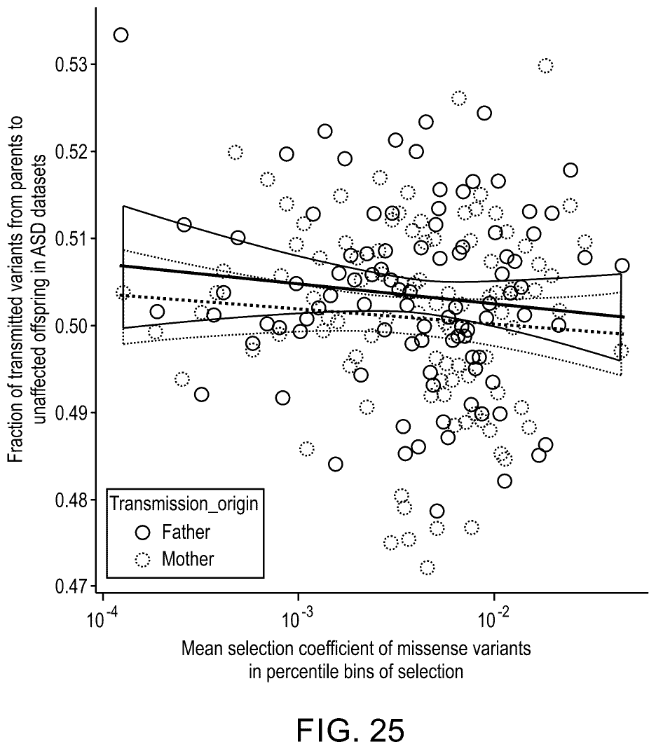

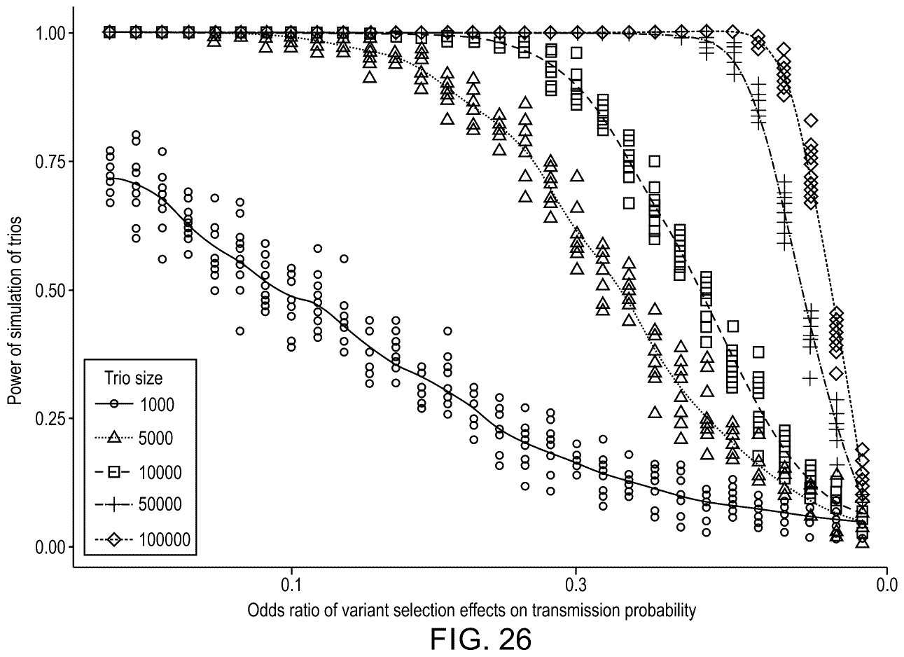

Imbalanced transmission of deleterious alleles among trios—In view of the finding that the impact on lifespan may be mediated by genes involved in developmental disorders, the question of whether selection burden correlates with allele transmission within families was additionally explored. With respect to this study, 877 trios were identified in the UKBB by kinship metrics and any family with mendelian errors or the family transmission rate beyond 0.4 and 0.6 were removed. As the trio size was small, significant correlation between allele transmission fraction and variant selection coefficient (FIG. 24) was not observed. The 5,097 trios with healthy offspring were obtained from the large-scale collection of autism trio datasets from the SPARK and ASC cohort to identify parent-offspring trios to quantify transmission from parents to children. To ensure the absence of bias due to ancestry or variant quality, data was restricted to non-Finnish Europeans and those rare variants with gnomAD allele frequency <0.01% (note in the case of parental transmission, there is no reliance on an assumption of Hardy-Weinberg proportions). Among the 4,033 retained trios, logistic regression analyses were run to evaluate the effects of variant selection pressure on their allele transmission from parents to normal offspring. A slight decrease in transmission among alleles with high estimated selection coefficients (FIG. 25) was observed. However, the p-values of logistic regressions are not significant (for alleles transmitted from father, the p-value is 0.9463 and from mother, the p-value is 0.282), likely due to the lack of power. Power analysis via simulation was then performed using UKBB samples and demonstrated that detection of moderate variant effects on allele transmission requires at least hundreds of thousands of trios (FIG. 26).

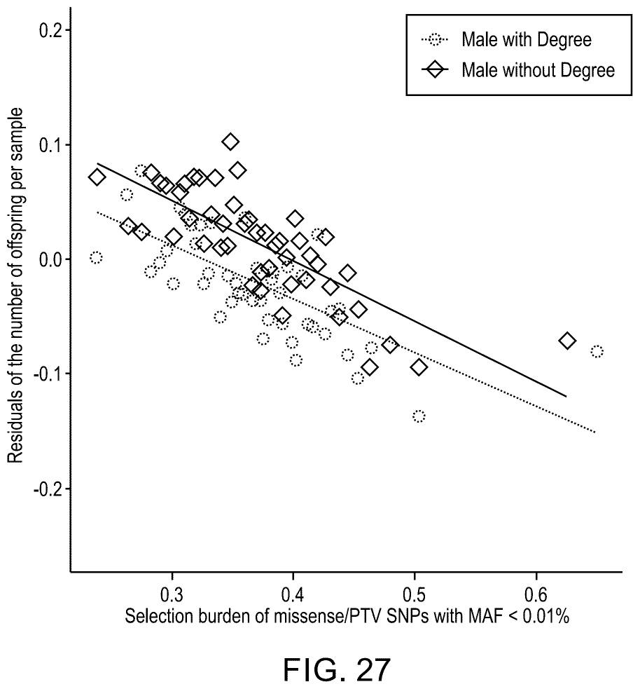

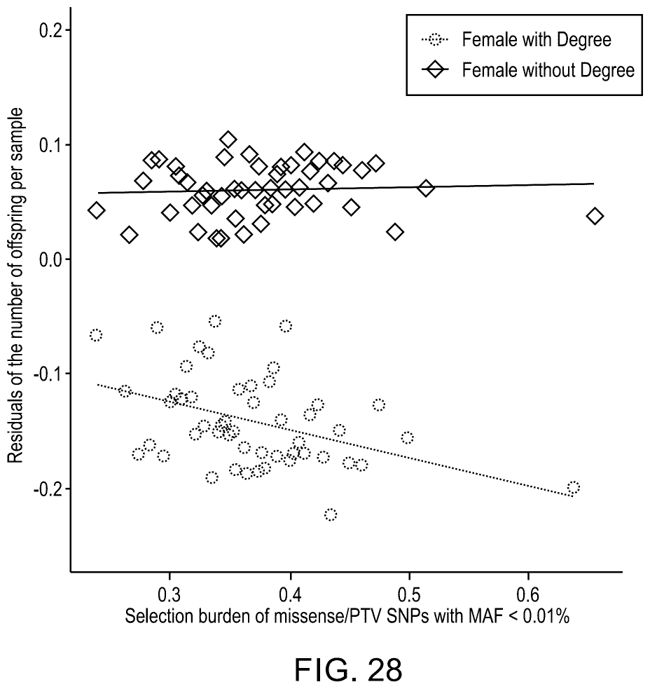

Selection burden reduced reproductive success in both males and females-Another major fitness component is number of offspring. PTV selection burden has been shown to be associated with reduced reproductive success of male samples in UKBB. Based on prior analyses, the techniques discussed herein were applied by incorporating deleterious missense variants into the multiplicative fitness measurement so as to further develop this work. Consistent with previous findings, it was found that the number of children decreases significantly with selection burden in male samples (effect size=−0.441, P=2.198e-29), while in female samples the effect is not significant (effect size=0.0199, P=0.561). It was estimated that reduced reproductive success due to selection burden potentially accounts for 26.8% of the total reduction in fitness expected due to purifying selection against burden of missense and PTVs (FIG. 27). When samples were stratified by whether they had a college degree (FIG. 28), it was found that selection burden still has negligible effects on reproduction in females without college degree (effect size=0.01955, P=0.627), but that it was significantly associated with fewer children among women with college degrees (effect size=−0.202, P=2.29e-3). On the other hand, there was no substantial effect of having a degree on male reproductive success (effect size=−0.428, P=4.16e-10 with degree; effect size=−0.454, P=3.754e-21 without degree).

### Methodology

Forward-time simulation of human population expansion-Forward-time simulations assuming neutral evolution were used to search for optimal parameters by fitting the simulated allele frequency spectrum (AFS) to that of the observed rare synonymous mutations in the human genome. The evolutionary history of human population was simplified into four stages of exponential expansion with different growth rates, assuming an initial effective population size is 10,000 as discussed in the following section. With reference to this subject matter, aspects of the forward time model are further discussed in U.S. Patent Publications 2022/0027388 and 2022/0028485, the disclosures of which are hereby incorporated by reference in their entirety for all purposes.

The Forward Time Simulation Model—As may be appreciated, the present frequency of a mutation is shaped by its evolutionary dynamics, with more deleterious mutations expected to be rarer. Over the course of evolution, new variants are constantly generated by mutation while others are removed by natural selection. If the human population size were constant, the allele frequencies of variants affected by the two forces would ultimately reach equilibrium. However, the human population size has grown exponentially, and this has impacted the frequencies of deleterious alleles. Therefore, forward time simulation was used to model the effects of these two forces on the distribution of allele frequencies of variants.

A forward-time population expansion model was simulated assuming neutral evolution and searched for optimal parameters by fitting the simulated allele frequency spectrum (AFS) to that of the observed rare synonymous mutations in human populations. The evolutionary history of the human population was modeled with four stages of exponential expansion with different growth rates, assuming an initial effective population size Ne0=10,000. The ratio between census population size and effective population size was denoted as r and a generation time of 30 years was assumed.

A burn-in period (about 3500 generations) was run with a small change in effective population size, with the population size change denoted as n. It was anticipated that this burn-in would mimic the bottleneck effect on human population during the ice age. As the time at the end of the burn-in period is unknown, this time was denoted as T1 and the effective population size at T1 as 10,000*n. The growth rate of the population expansion during the burn-in period is n\hat{ }(1/3500).

The human census population size in 1400 CE is estimated to be about 360 million. The number of generations between T1 and 1400 CE is (1400−T1)/30, denoted as g. The population growth rate for this stage between T1 and 1400 CE is (3.6/r/n) \hat{ }(1/g).

By 1700 CE, the world census population size increased to about 620 million. The number of generations between 1400 CE and 1700 CE is 10, thus the growth rate is (6.2/3.6)\hat{ }(1/10).

At 2000 CE, the census population size is 6.2 billion, 10 times that of 1700 AD. For this stage of 10 generations, the growth rates become (10)\hat{ }(1/10).

Because these estimated census population sizes are fixed and it is assumed that the effective population size is a fixed proportion of the census size, three parameters are estimated: r, the ratio of the effective to census population sizes, n, the census population size at a time T1 in the past, and g, the number of generations between T1 and the year 1400 CE. In total, the simulation of this model aims to optimize three demographic parameters (r, g, and n).

Because rare mutations are the focus of the presently described studies, simulation was initialized with all chromosomes fixed for the ancestral allele. Then, in each generation, the population size is expanded according to the growth rate and chromosomes are sampled uniformly at random from the previous generation. De novo mutations were then placed onto these chromosomes with rates given by de novo mutation rates, which were obtained from three large parental-offspring trio datasets (totaling 8,071 trios) with whole genome sequencing. De novo mutations that mapped to intergenic regions were obtained and the mutation rates estimated for each of the 192 trinucleotide context configurations. The Spearman correlation between the estimates of mutation rates with gnomAD mutation rates was determined to be 0.9991, but the estimates tended to be generally lower at the CpG sites.

Due to the substantial impact of methylation levels at CpG sites on the mutation rates, it was determined to be appropriate to account for methylation levels modeling CpG transition mutation rates. First, the impact of methylation levels on CpG mutation rates was evaluated by using whole genome bisulfite sequencing data. The methylation data for each CpG island were extracted and averaged across 10 embryonic stem cell (ESC) samples. CpG islands were then separated into 10 bins according to 10 methylation levels. The number of CpG sites, as well as the observed number of CpG transition variants, falling in each methylation bin were counted in both intergenic and exonic regions. The expected number of transition variants at CpG sites in each methylation bin is computed as the total number of CpGTi variants multiplied by the fraction of CpG sites in that methylation bin. It was observed that there is about a fivefold change in ratios of observed/expected numbers of CpGTi mutations from high and low methylation levels. Thus, CpG sites were classified into two categories: high methylation if the averaged methylation level is greater than 0.5 and low methylation if the averaged methylation level is less than or equal to 0.5, and the de novo mutation rates were computed for each of the 8 CpGTi tri-nucleotide contexts for high and low methylation levels. Averaging across the 8 CpGTi tri-nucleotide contexts, the CpGTi mutation rates were obtained, 1.01e-07 for high methylation and 2.264e-08 for low methylation, approximately an order of magnitude difference.

De novo mutation rates were averaged across 56 tri-nucleotide contexts with transition mutations at non-CpG sites (nonCpGTi) and across 128 tri-nucleotide contexts with transversion mutations (Tv) to produce a nonCpGTi mutation rate 5.552238e-09 and a Tv mutation rate 2.034197e-09, respectively. One hundred thousand independent sites were then simulated using these mutation rates according to the demographic model above, and allele frequency spectra were generated from the simulated sites separately for nonCpGTi and Tv. For CpGTi mutations, 100,000 sites were separately simulated using mutation rates at the high and low methylation levels derived above. The two simulated AFSs were merged by applying the known proportions of high or low methylation sites in the human genome as weights.

Fitting the AFS of genome Aggregation Database (gnomAD) exome sequencing data—To find the best fitting parameters of the demographic model, a grid search was performed across the parameters g∈(330, 350, 370, 400, 430, 450, 470, 500, 530, 550), r∈(20, 25, 30, . . . , 100, 105, 110) and n∈(1.0, 2.0, 3.0, 4.0, 5.0). For each combination of the parameters, a human population was simulated to present day, then 1,000 sets of 246,000 chromosomes were randomly sampled, corresponding to the sample size of gnomAD exomes. The expected AFS was then generated by averaging across the 1,000 sampled sets.

Human exome polymorphism data from Genome Aggregation Database v2.1.1 and the allele frequency spectrum of rare synonymous variants was generated. A likelihood ratio test was applied to evaluate the goodness-of-fit of simulated AFS to the gnomAD AFS of synonymous variants across the three mutational classes to determine the optimal parameters. The optimal parameter combination was achieved when g=530, r=30, and n=2.0.

Application of the Forward Time Simulation Model-Because the focus was on rare mutations, the simulation was initialized with all chromosomes fixed for the ancestral allele. Then, in each generation, the population size changes according to the growth rate and chromosomes were sampled uniformly at random from the previous generation. De novo mutations were then placed onto these chromosomes according to the de novo mutation rates, which were derived from large parental-offspring trio datasets with whole genome sequencing for a total of 8071 trios when merged. The de novo mutations mapped to the intergenic regions were obtained and the mutation rates derived for each of the 96 trinucleotide context configurations.

One hundred thousand independent sites were simulated and allele frequency spectra generated separately for each of the three major classes of mutations, i.e., transition mutation at CpG sites (CpGTi), transition mutation at non-CpG sites (nonCpGTi) and transversion mutations (Tv). For CpGTi, sites were separately simulated with high and low methylation levels, due to the impact of CpG methylation on mutation rate, and then the two AFSs were merged by applying the known proportions of high or low methylation sites in the human genome as weights. For each combination of parameters, a human population to present day was simulated and 1,000 sets of 246,000 chromosomes were randomly sampled, corresponding to the sample size of gnomAD exomes. The expected AFS was then generated by averaging across the 1,000 sampled sets.

Human exome polymorphism data was downloaded from the genome Aggregation Database v2.1.1 and the allele frequency spectrum of rare synonymous variants was generated. The likelihood ratio test was applied to evaluate the fitness of simulated AFS to the gnomAD AFS across the three mutational classes to determine the optimal parameters.

Simulation with selection effects—With an optimized population history derived as discussed in the preceding, selection was incorporated into the simulations across a grid of selection coefficients, s, in {0, 0.0001, 0.0002, . . . , 0.8, 0.9}. Then, for each generation, after sampling chromosomes and de novo mutations, mutations were randomly purged according to their selection coefficient. For each of the 36 selection coefficients and each of 192 mutation rates, 1,000 sets of 500,000 genomes were randomly sampled, corresponding to the sample size of gnomAD, Topmed, and UK Biobank 200K. The sampled AFSs were then aggregated across the 192 de novo mutation rates for each selection coefficient. As the selective pressure against mutations rises, variants are expected to be progressively more depleted relative to neutral variants. With the simulated AFS under various levels of selection, “depletion” was defined as measuring the proportion of variants wiped out by purifying selection compared to the scenario under neutral evolution (without selection):

$$Depletion = 1 - \frac{♯ variants with selection}{♯ variants with out selection} \qquad ( 1 )$$

Depletion values were generated for each of the 36 selection coefficients to create a selection depletion curve, which were interpolated to obtain the estimated selection coefficient given any depletion value.

Estimating depletions and selection coefficients for loss-of-function mutations—In certain of the studies discussed herein, the depletion of protein truncating variants (PTVs) was calculated. Due to the difficulty in computing the expected number of frameshifts in a gene, techniques were employed to focus on stop-gained variants and splicing variants, denoted as loss-of-function (LOF) mutations. Variant allele frequency data from approximately 500,000 individuals was collated and merged, including 122,439 gnomAD exomes and 70,304 gnomAD WGS (after removing Topmed samples), 65K Topmed WGS, and 200K UK Biobank exomes. The number of stop-gain and splicing variants in each gene in the merged dataset was taken to be the observed number of LoF mutations. To calculate the expected number of LoF mutations, constraint metrics from gnomAD were downloaded and employed. Because the expected number of LoF mutations is based on the mutation rate, it was assumed that the ratio of expected LoF mutations to synonymous variants in the merged dataset would be similar to that observed in gnomAD; thus:

$$\# exp LoF = \# obs Syn * \frac{\# exp LoF of gnomAD}{\# exp Syn of gnomAD} \qquad ( 2 )$$

The depletion metric is then:

$${depletion}_{LoF} = 1 - \frac{\# obs LoF}{\# exp LoF} \qquad ( 3 )$$

If a depletion was observed outside of the interval [0,1] it was forced to the closest boundary.

Based on this approach, the selection coefficient of LoF mutations in each gene can be estimated by identifying the selection coefficient that corresponds to the depletion value of those mutations.

Estimating depletions and selection coefficients for missense variants—For each gene of at least 100 amino acids in length and with at least 5 observed synonymous variants in gnomAD, sites in the gene were binned into PrimateAI-3D (PAI) deciles and the number of the observed missense variants falling in each bin was counted. The expected number of missense mutations in each bin was computed with similar logic to LoF mutations, although the depletion metric requires the expected number of missense mutations to be divided by 10, to correspond to the 10 deciles:

$${depletion}_{Mis} = 1 - \frac{\# obs Mis in each bin}{\# exp Mis / 10} \qquad ( 4 )$$

$$where$$

$$\# exp Mis = \# obs Syn * \frac{\# exp Mis of gnomAD}{\# exp Syn of gnomAD} \qquad ( 5 )$$

For each bin and each gene, the median PAI score of that bin was computed and a smoothing spline fit between the PAI and the depletion. Then, a selection coefficient was estimated for each missense mutation by computing the depletion predicted based on its PAI score, and finally a selection coefficient was inferred using the mapping of depletion to selection based on the simulations.

Score percentiles (e.g., PrimateAI-3D score percentiles in the present example) were re-calibrated before computing the depletion metrics to account for the noise in the observed PAI percentiles. The observed PAI percentiles were first modeled as discrete-uniformly distributed true PAI percentiles overlayed with Gaussian noise. An assumption was made that true PAI percentiles are discrete-uniformly distributed over (0,1], e.g., they take 100 values, [0.01, 0.02, . . . , 0.99, 1.00]. The observed PAI percentiles center at the true PAI percentiles with some noise terms which follows normal distribution with empirical standard deviation 0.15.

$$obsPAI ∼ truePAI + e , e ∼ N ( 0 , sd = 0.15 ) \qquad ( 6 )$$

The distribution of observed PAI percentiles is the discrete uniform distribution with Gaussian noise added. The density distribution and its cumulative distribution function (CDF) of observed PAI percentiles were then generated. From this CDF, the cumulative probability was divided into 100 intervals, and new quantiles for observed PAI percentiles were generated.

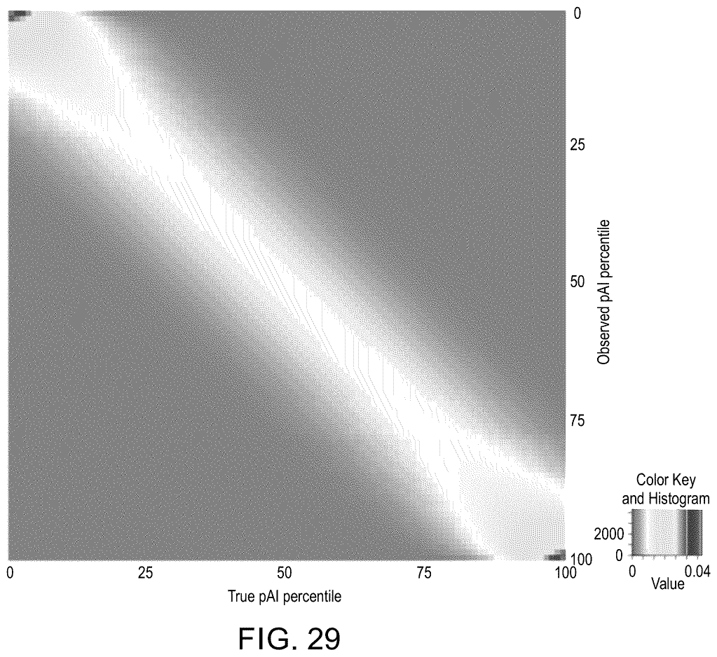

For each gene, a 100×100 probability matrix was generated where each value represents the probability that a variant with the observed PAI percentile actually comes from one of the true PAI percentiles, as shown in the heatmap (FIG. 29). Each row of this 100×100 probability matrix was normalized to sum to one.

The true PAI percentiles were divided into 10 bins, i.e., for each row of the heatmap matrix, the probabilities were merged from 1%-10% into the 1st bin (the first 10 columns of the recalibrated heatmap), 11%-20% into the 2nd bin (the next 10 columns of the recalibrated heatmap), . . . , etc. Thus, the value of each of 10 bins represented the probability a variant might come from that true PAI percentile bin. Given one variant of that gene that has an observed PAI percentile, e.g., x %, corresponding to the x-th row of the recalibrated heatmap, the probability can be obtained that this variant might fall within each of the 10 true PAI percentile bins, denoted as variant contribution to each bin. The expected number of missense variants falling within each of the 10 bins is the sum of the variant contribution to that bin across all the observed missense variants in that gene. In this way, the recalibrated expected number of missense variants falling within each of the 10 bins for a gene were obtained so as to allow calculation of the depletion metric for each missense bin.

By recalibrating the scores (e.g., PrimateAI-3D score in the present example), the noise distribution was modeled in percentiles of the scores and the noise reduced in the predicted depletion metrics, which in terms mitigated the effects of noise on estimation of selection coefficients in missense variants.

Survival analysis using selection burden-Cox proportional hazards regression was applied with right censoring to test for an association between survival and selection burden. To control for ancestry, the analyses were restricted to UK Biobank participants with ‘Caucasian’ genetic ethnic grouping based on principal component analysis (PCA) (UKB Data-Field 22006) and who were born between 1934 and 1970 with parental lifespan ≥20 years. In total, 362,776 participants were obtained, including 165,147 male and 197,629 female participants. Probands who were adopted by their parents were excluded from parental lifespan analysis (n=2,703).

A regression model was applied controlling for age at enrollment, proband sex, and genetic principal components:

$$Lifespan ∼ burden + age + {age}^{2} + birth . cohort + sex + PC 1 … PC 40 \qquad ( 7 )$$

where age is the individual's age at enrollment. Sex information was included when male and female samples were combined for analyses. A categorical variable (birth.cohort) was incorporated in the model for all possible 5-year birth cohort windows (for example, born between 1940 and 1945, 1946 and 1950, and so on). PC1 . . . PC40 are pre-computed ancestry principal components for each UK Biobank participant and acquired via the UK Biobank data showcase. The regression models were run for proband lifespan, fathers' lifespan, mothers' lifespan, and the combined parental lifespan by averaging imputed father's and mother's lifespan for each proband. All Cox regressions were performed in R with the ‘survival’ package.

The measure of variance (R²) explained for censored survival time proposed by Royston et al. was computed to evaluate the effect of selection burden on proband survival. The baseline R² was generated by performing Cox's regressions with covariates only and then calculated R² incorporating selection burden in the Cox's regressions. The increased amount of R² compared to the baseline R² indicates the effect size of selection burden on survival time prediction. These analysis metrics were applied to father and mother survival, respectively.

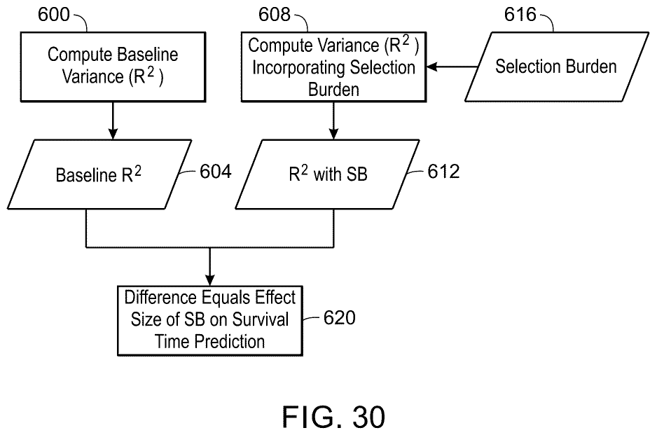

With the preceding in mind, and turning to FIG. 30, a process flow of steps, suitable for being performed by a processor-based system (e.g., desktop or notebook computer, workstation, or specialized sequencing device) are depicted. Such steps may be performed as part of a treatment or examination of a subject or patient or as part of a study protocol. Intermediate or final outputs or results of such steps may be provided as part of a report or diagnosis which may be stored on a processor-based system, displayed for review by such a system (such as on a user interface of such a system), printed for storage or review, or may be used as part of a decision making process based on the analysis, such as by displaying one or more selectable treatment options for the subject or by automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

With this in mind, and turning to FIG. 30, aspects of the above description are illustrated as steps of a process that may be used for quantifying an effect of selection burden on a survival time prediction. In this example, a baseline variance 604 is computed (step 600) without taking into account selection burden and a separate variance 612 is computed (step 608) that incorporates selection burden 616. By taking a difference between the baseline variance 604 and the variance incorporating selection burden, a quantification 620 of the effect of the selection burden 616 on estimated survival time can be obtained. Such data may be displayed for review on a user interface of a processor-based system, and/or or may be used as part of a decision-making process, such as for identifying one or more treatment options, automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

For the gene-burden survival analysis, the counts of LoF mutations in each gene were used as rare variant burden to test whether some genes' burdens are associated with either proband or parental survival. Genes with fewer than 10 PTVs in non-censored individuals were excluded from survival analysis to avoid the bias of Cox's model. LoF and missense variants with the top 50% of PrimateAI-3D scores were counted in each gene as gene burden for survival analyses. The p-values of gene burdens were adjusted for multiple testing among 15,000 genes and three survival scenarios, i.e., proband, paternal, and maternal survival. For each gene's Cox regressions, increased variance explained for censored survival time was also computed.

For the gene-set burden survival analysis, 315 gene panels (at least 5 genes in a panel) with expert curation were downloaded from PanelApp and the selection burden was generated using multiplicative model restricting to genes in each panel. Cox regressions using these gene-set burdens were run for proband, paternal, and maternal survival, separately.

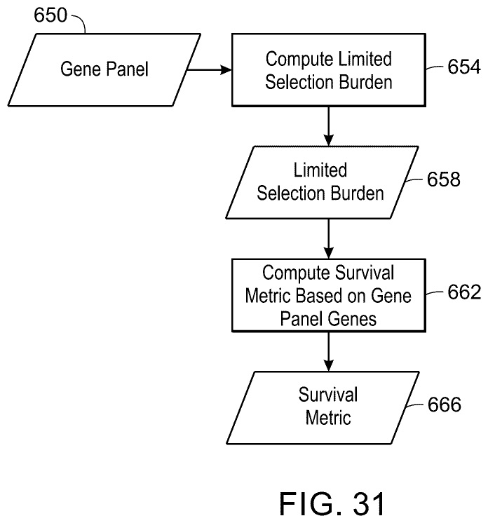

With the preceding in mind, and turning to FIG. 31, a process flow of steps, suitable for being performed by a processor-based system (e.g., desktop or notebook computer, workstation, or specialized sequencing device) are depicted. Such steps may be performed as part of a treatment or examination of a subject or patient or as part of a study protocol. Intermediate or final outputs or results of such steps may be provided as part of a report or diagnosis which may be stored on a processor-based system, displayed for review by such a system (such as on a user interface of such a system), printed for storage or review, or may be used as part of a decision making process based on the analysis, such as by displaying one or more selectable treatment options for the subject or by automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

With this in mind, and turning to FIG. 31, aspects of the above description are illustrated as steps of a process that may be used for quantifying a survival metric for a subject. In this example, a gene panel 650 is acquired for the subject and a selection burden 658 limited to the genes assessed by the gene panel 650 is computed (step 654). Based on the limited selection burden 658, a quantitative survival metric 666 may be computed (step 662) that is based on the genes associated with the gene panel 650. Such a quantitative survival metric may be displayed for review on a user interface of a processor-based system, and/or or may be used as part of a decision-making process, such as for identifying one or more treatment options, automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

Linear modelling of imputed lifespan—To produce interpretable estimates of the impact of selection burden in terms of years of life, the lifespans of living individuals were imputed using actuarial tables from the UK Office of National Statistics and standard linear regressions were run using the same covariates as the survival analysis:

$$Lifespan ∼ burden + age + {age}^{2} + birth . cohort + gender + PC 1 … PC 40 \qquad ( 8 )$$

The effect of covariates was regressed out from the imputed lifespan and the regression was run on the residuals of lifespans. This produced the average number of years of lifespan lost due to each mutation.

Multistate illness-death modeling-Multistate modeling as implemented in the R package mstate was used to fit illness-death models. Specifically, individuals were allowed to transition from a healthy state at birth to a disease state, from a healthy state to a dead state, and from a disease state to a healthy state. These models appropriately handle right censored disease status and death status and correctly adjust baseline hazard rates for the fact that disease status may be censored due to dying (as opposed to not having the disease by the last observation date). To ensure that there was sufficient power, diseases were restricted to those having prevalence above 0.1% in the UKBB probands. Then, for each disease, the illness-death model was fit using Cox Proportional hazards regression with the formula:

$$Survival ( status , time ) ∼ burden + age + gender + PC 1 … PC 40 \qquad ( 9 )$$

where age is the individual's age at enrollment.

To compute the years lost to a disease, Cox proportional hazards modeling and mstate were used to compute baseline survival curves for the transitions from the healthy state to death (S(t)) and from the disease state to death (S*(t)). A metric of years of life lost based on expected years of life left given age of onset of the disease was then used, because it is robust to right censored data, in contrast to commonly used estimators based on the age at death of an individual with a given disease. Specifically, at age a, an individual's expected years remaining is:

$$E ( a ) = {\int }_{a}^{c} S ( t ) dt / S ( a ) \qquad ( 10 )$$

if they are healthy and:

$${E}^{*} ( a ) = {\int }_{a}^{c} {S}^{*} ( t ) dt / {S}^{*} ( a ) \qquad ( 11 )$$

if they have disease, where c is a constant indicating a maximum lifespan (chosen to be 80). So, the expected years of life lost for individual i diagnosed at age aᵢ is:

$$L ( {a}_{i} ) = E ( {a}_{i} ) - {E}^{*} ( {a}_{i} ) \qquad ( 12 )$$

and finally the average years of life lost due to the disease is:

$$YLL = \frac{1}{n} {\sum }_{i = 1}^{n} L ( {a}_{i} ) \qquad ( 13 )$$

where the sum ranges over all people diagnosed with the disease. The integrals over the survival were numerically estimated based on the piece-wise constant baseline hazard ratios inferred by mstate by noting that the survival function is the exponential of the cumulative hazard function.

Testing burden disease association with curated gene sets—To determine if the signal of association between burden and disease was specific, a PanelApp gene set was employed. In this gene set, 315 gene panels with expert curation from PanelApp were used. For the gene burden studies, nominal burden p-values were downloaded. Reasoning that there may be some signal in hits that did not reach genome-wide significance, but not wanting to include too much noise, data was restricted to the top 30 genes by association p-value, as long as the p-value was less than 0.01. In cases where p-values were unavailable, a random subset of 30 genes were selected. For PanelApp panels, every gene available in the panel was used.

The multistate modeling was then run again with restricting to each gene set. To compare the associations between gene sets, Z-scores of the −log 10 (p-value), normalized by gene set, were computed for each disease. Thus, each gene set could be treated roughly equivalently, regardless of gene set size or association strength, and gene sets that were strongly associated with only a small number of diseases could be identified. For visualization, analysis was initially restricted to gene sets with at least one disease having Z>5, indicating at least one strongly associated disease, and the top 3 diseases by p-value were selected for that gene set. Diseases were then clustered via the correlation between their Z scores.

Transmission rates of deleterious alleles among siblings and parent-offspring trios—Sib pairs were identified in UKBB by first using the provided estimates of IBS0 and Kinship provided by UKBB and restricting to pairs of individuals with Kinship between 0.23 and 0.27 and IBS0>0.002. These pairs were further filtered to ensure that both members were white British.

Given an allele at frequency x and a mutation rate u, under the assumption of Hardy-Weinberg proportions in the parental generation a probability that one sibling of a sibling pair is heterozygous for a variant given that the other sibling is heterozygous for that variant can be computed. This probability assumes no developmental selection and perfect sequencing quality. Most variant callers are conservative when calling heterozygous sites, especially from rare variants. Thus, a term (1-m) was additionally included where m is the rate of missingness of heterozygotes. Finally, a factor of (1-s) was included to capture the effect of selection, resulting in the final formula for the probability that one sibling is heterozygous conditional on the other sibling being heterozygous:

$${ρ}_{het} ( x , μ , m , s ) = {ρ}_{het , neutral} ( 1 - m ) ( 1 - s ) \qquad ( 14 )$$

This can be used as part of a binomial likelihood to estimate the unknown parameters.

For allele frequencies, non-Finnish European allele frequencies from gnomAD were used and data restricted to variants with allele frequencies above 0. The mutation rate was then first fit using synonymous variants. To fit missense variants, it was assumed that the effect on selection during development is proportional to the estimated selection coefficient, modulated by a fraction f. Then, the parameters m and f were fit using all missense variants in UKBB sib pairs with s>1e-4. Following this model fitting, a model was also fit fixing f=0, representing a null model where there is no selection during development, and performed a likelihood ratio test with one degree of freedom to compare the two models.

Linear modelling of number of offspring—The UK Biobank participants with ‘Caucasian’ genetic ethnic grouping who were born between 1934 and 1970 with the number of children between 0 and 10 were again used for analysis. In total, 379,613 participants were obtained, including 173,733 male and 205,880 female participants. Linear models on selection burden were run using the formula below separately for male and female samples. The covariates are similarly defined as in the lifespan models.

$$Number of children ∼ burden + age + {age}^{2} + birth . cohort + PC 1 … PC 40 \qquad ( 15 )$$

The effect of covariates from the number of offspring was regressed out and the regression run on the residuals of number of offspring stratifying males or females based on obtaining college degree or not.

UK Biobank data preprocessing-UK Biobank is a prospective study of over 500,000 participants aged 40-69 years recruited from 2006 to 2010 in the UK. To collate phenotypes for all individuals in UK Biobank for use in the studies discussed herein, bulk phenotype files were downloaded from the UK Biobank data showcase. To collate variants for all 450K individuals with whole-exome sequencing, the GRCh38-aligned population-level variant call format files were downloaded from the UK Biobank (UK Biobank field 23156). Variants passing quality filters were then annotated using VEPv102, gnomAD allele frequency and PrimateAI-3D scores. For all the UK Biobank analyses discussed herein, only autosomal variants with a gnomAD-specific allele frequency <1×10⁻⁴ were retained.

Calculating selection burden for UK Biobank participants-A multiplicative model of fitness was used to calculate individual level selection burden:

$${B}_{i} = 1 - {\prod }_{l = 1}^{L} {G}_{il} ( 1 - {s}_{l} ) \qquad ( 16 )$$

where Bᵢ is the burden of individual i, Gᵢₗ indicates whether individual i carries the minor allele at variant l, and sₗ is the selection coefficient of variant l.

To compare the distribution of selection burden across different ethnicities, including Europeans, Asians and Africans, any variants with the maximum of gnomAD allele frequency among any subpopulations >0.01% were removed to reduce artifacts due to variable allele frequency between populations. Different selection coefficient thresholds were then applied to filter variants when computing the selection burden or rare variant burden (number of deleterious variants).

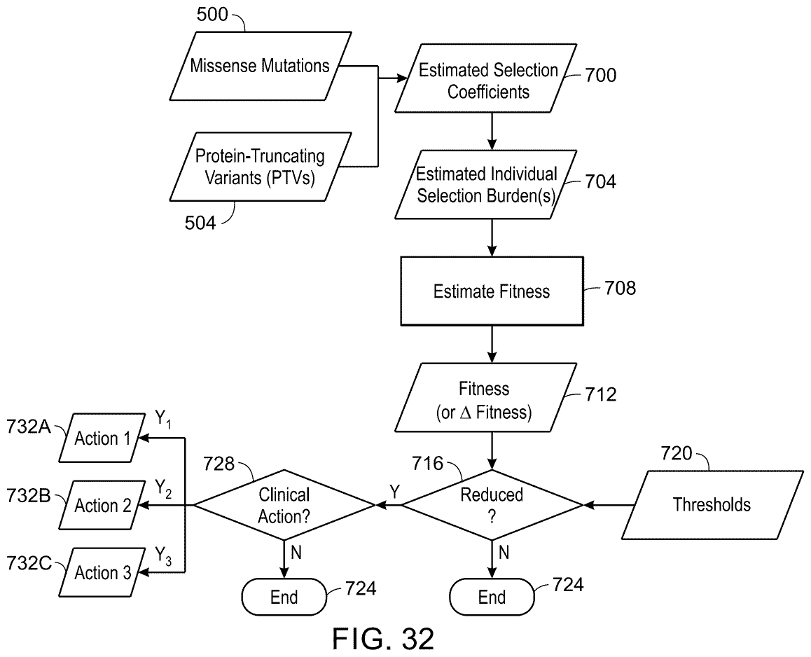

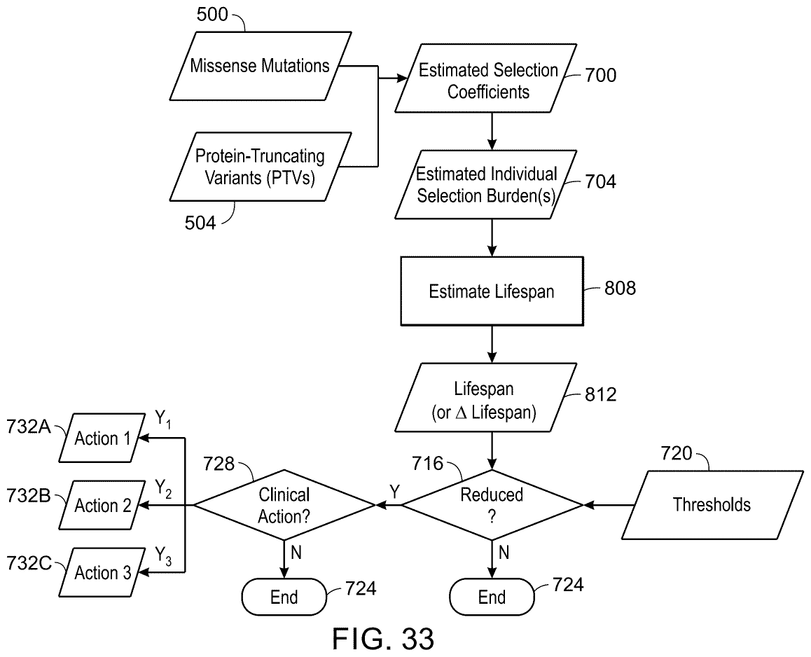

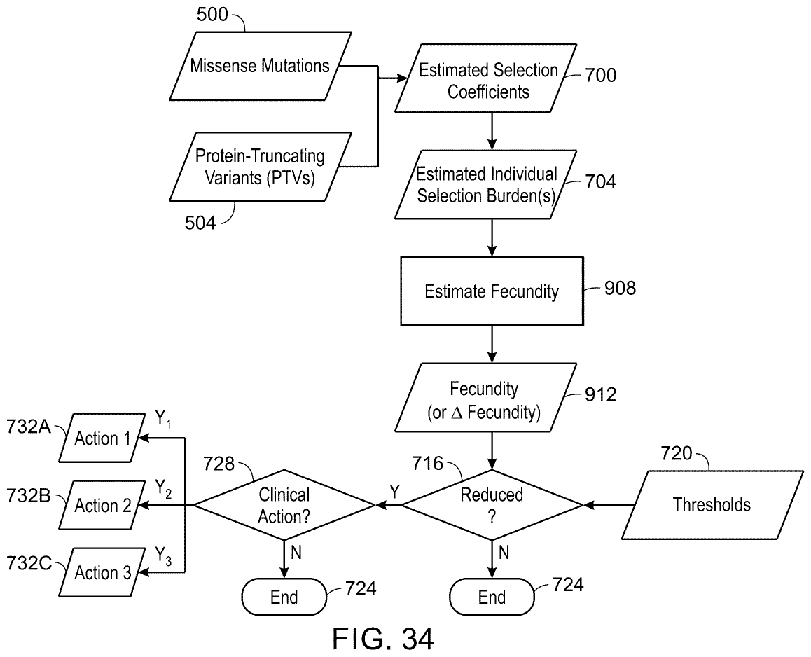

With the preceding discussion and examples in mind, and turning to FIGS. 32, 33, and 34, respective process flows of steps are proved that are, suitable for being performed by a processor-based system (e.g., desktop or notebook computer, workstation, or specialized sequencing device). Taking into consideration the preceding discussion of techniques and approaches, FIG. 32 describes steps useful in estimating a quantitative measure for fitness and providing one or more clinical action options based on the fitness metric, while FIG. 33 describes a similar process flow in the context of lifespan and FIG. 34 describes a similar process flow in the context of fecundity.

As will be appreciated, such steps may be performed as part of a treatment or examination of a subject or patient or as part of a study protocol. Intermediate or final outputs or results of such steps may be provided as part of a report or diagnosis which may be stored on a processor-based system, displayed for review by such a system (such as on a user interface of such a system), printed for storage or review, or may be used as part of a decision making process based on the analysis, such as by displaying one or more selectable treatment options for the subject or by automatically selecting and recommending one or more such treatment options. Such options may include, but are not limited to clinical research options (including automatic enrollment in research studies, support groups, and so forth), pharmaceutical treatment options, lifestyle changes or recommendations, and so forth.

With this in mind, and turning to FIG. 32, aspects of the above description are illustrated as steps of a process that may be used for quantifying a Fitness metric for a subject. In this example, missense mutations 500 and protein truncating variants 504 for genes within the human genome are used, as discussed herein to calculate estimated selection coefficients 700 for each missense mutation 500 and protein truncating variant 504. The estimated selection coefficients 700 may in turn be used to calculate estimated individual selection burdens 740 for a subject, such as based upon a set of genes assessed for the subject using one or more gene panels. Based on the estimated individual selection burden 704, a quantitative fitness metric 712 or fitness differential metric may be estimated (step 708).

Based on the fitness metric 712 (or differential) a determination (decision block 716) may be made as to whether the fitness of the individual is reduced, such as relative to one or more specified thresholds 720. If the fitness of the individual is not reduced or is not reduced in excess of a threshold 720, the process may terminate (end step 724). If the fitness of the individual is reduced, either absolutely or in the context of the threshold(s) 720, the process may proceed to decision block 728 at which a determination may be made as to whether clinical action is needed. If a determination is made that no clinical action is needed, the process may terminate (end step 724). If a determination is made that a clinical action is needed at decision block 728, one or more clinical action recommendations (e.g., actions 732A, 732B, and 732C) may be generated and presented to a reviewer for review or selection, such as to generate a treatment plan or counseling guideline. While FIG. 32 is presented in the context of a clinical decision-making process so as to provide a useful real-world context for explanation, in other embodiments the action(s) in question may be non-clinical in nature, such as insurance options or pricing, financial or investment options or alternatives, lifestyle options, and so forth.

Turning to FIG. 33, a similar process is depicted but in the context of lifespan or change in lifespan. In this example, based on the estimated individual selection burden 704, a quantitative lifespan metric 812 or lifespan differential metric may be estimated (step 808). Based on the lifespan metric 812 (or differential) a determination (decision block 716) may be made as to whether the lifespan of the individual is reduced, such as relative to one or more specified thresholds 720. If the lifespan of the individual is not reduced or is not reduced in excess of a threshold 720, the process may terminate (end step 724). If the lifespan of the individual is reduced, either absolutely or in the context of the threshold(s) 720, the process may proceed to decision block 728 at which a determination may be made as to whether clinical action is needed. If a determination is made that no clinical action is needed, the process may terminate (end step 724). If a determination is made that a clinical action is needed at decision block 728, one or more clinical action recommendations (e.g., actions 732A, 732B, and 732C) may be generated and presented to a reviewer for review or selection, such as to generate a treatment plan or counseling guideline. As with the discussion of FIG. 32, although the actions presented in the present discussion of FIG. 33 are presented in the context of a clinical decision-making process, in other embodiments the action(s) in question may be non-clinical in nature, such as insurance options or pricing, financial or investment options or alternatives, lifestyle options, and so forth.

Similarly, and turning to FIG. 34, a further process is depicted but in the context of fecundity or change in fecundity. In this example, based on the estimated individual selection burden 704, a quantitative fecundity metric 812 or fecundity differential metric may be estimated (step 808). Based on the fecundity metric 812 (or differential) a determination (decision block 716) may be made as to whether the fecundity of the individual is reduced, such as relative to one or more specified thresholds 720. If the fecundity of the individual is not reduced or is not reduced in excess of a threshold 720, the process may terminate (end step 724). If the fecundity of the individual is reduced, either absolutely or in the context of the threshold(s) 720, the process may proceed to decision block 728 at which a determination may be made as to whether clinical action is needed. If a determination is made that no clinical action is needed, the process may terminate (end step 724). If a determination is made that a clinical action is needed at decision block 728, one or more clinical action recommendations (e.g., actions 732A, 732B, and 732C) may be generated and presented to a reviewer for review or selection, such as to generate a treatment plan or counseling guideline.

As discussed in preceding examples, fitness, lifespan, and/or fecundity metrics as discussed with respect to FIGS. 32, 33, and 34 may be displayed for review on a user interface of a processor-based system, and/or or may be used as part of a decision-making process, such as for identifying one or more treatment options, automatically selecting and recommending one or more such treatment options (e.g., actions 732). Such options may include, but are not limited to clinical research options (including automatic enrollment in research studies, support groups, and so forth), pharmaceutical treatment options, lifestyle changes or recommendations, and so forth.

A Neural Network Pathogenicity Classifier-Reference is made herein to classification networks capable of generating scoring useful for estimation of selection coefficients and/or depletion metrics and curves, an example of which is the PrimateAI-3D (PAI) network discussed herein. By way of example and useful context, aspects of such a system are discussed below so as to illustrate aspects of such a system. In particular, the following discussion includes aspects related to the training and use of neural networks, including convolutional neural networks, which may be used to implement certain of the analytics discussed below, such as the generation of variant pathogenicity scores or classifications and the derivation of useful clinical analytics or metrics based on such pathogenicity scores or classifications. To streamline discussion, a baseline knowledge of such neural networks is presumed in describing the present techniques. With further reference to this subject matter, aspects of the PAI network are discussed in U.S. Patent Publications 2022/0027388 and 2022/0028485 as well as in U.S. application Ser. Nos. 17/968,285, 17/968,723, and 17/968,766, the disclosures of which are hereby incorporated by reference in their entirety for all purposes.

Regarding machine-learning based pathogenicity classifiers, deep neural networks are a type of artificial neural networks that use multiple nonlinear and complex transforming layers to successively model high-level features. Deep neural networks provide feedback via backpropagation which carries the difference between observed and predicted output to adjust parameters. Deep neural networks have evolved with the availability of large training datasets, the power of parallel and distributed computing, and sophisticated training algorithms.

Convolutional neural networks (CNNs) and recurrent neural networks (RNNs) are components of deep neural networks. Convolutional neural networks may have an architecture that comprises convolution layers, nonlinear layers, and pooling layers. Recurrent neural networks are designed to utilize sequential information of input data with cyclic connections among building blocks like perceptrons, long short-term memory units, and gated recurrent units. In addition, many other emergent deep neural networks have been proposed for limited contexts, such as deep spatio-temporal neural networks, multi-dimensional recurrent neural networks, and convolutional auto-encoders.

In the present context, such machine learning approaches may be used to score or rank variants for a given gene. By way of example, the techniques employed in the PrimateAI-3D (PAI) classifier as referenced herein utilize a deep learning residual network which is configured to receive as an input the amino acid sequence flanking a variant of interest and the orthologous sequence alignments in other species. The presently described deep learning network was trained to extract features directly from the primary sequence. In certain implementations, to provide the network with information about protein structure two separate networks may be trained to learn, respectively, secondary structure and solvent accessibility from sequence alone. These may be incorporated as sub-networks in the larger deep learning network to predict effects on protein structure. In one example of an implementation, the deep learning network for pathogenicity prediction contains 36 total convolutional layers, including 19 convolutional layers for secondary structure and solvent accessibility prediction sub-networks, and 17 for the main pathogenicity prediction network which takes as input the results of the secondary structure and solvent accessibility sub-networks. Using sequence as a starting point avoids potential biases in protein structure and functional domain annotation, which may be incompletely ascertained or inconsistently applied.

The referenced PAI techniques leverage variation from non-human primates (e.g., chimpanzee, bonobo, gorilla, orangutan, rhesus, and marmoset) that contributes over 300,000 unique missense variants that are non-overlapping with common human variation and largely represent common variants of benign consequence that have been through the sieve of purifying selection. This greatly enlarges the training dataset available for machine learning approaches. The accuracy of the deep learning classifier scales with the size of the training dataset, and variation data from each of six primate species independently contributes to boosting the accuracy of the classifier. The large number and diversity of extant non-human primate species, along with evidence showing that the selective pressures on protein-altering variants are largely concordant within the primate lineage, suggests systematic primate population sequencing as an effective strategy to classify the millions of human variants of unknown significance that currently limit clinical genome interpretation. Further, common primate variation provides a clean validation dataset for evaluating existing methods that is completely independent of previously used training data, which has been hard to evaluate objectively because of the proliferation of meta-classifiers.

With respect to the pathogenicity prediction model component of the PAI neural network model, semi-supervised deep convolutional neural network (CNN) models were developed to predict pathogenicity of variants. The input features to the models include protein sequences and conservation profiles flanking variants and depletion of missense variants in specific gene regions. The changes caused by variants to secondary structure and solvent accessibility were also predicted by deep learning models and this was integrated into the pathogenicity prediction model. Predicted pathogenicity in one such implementation is on a scale from 0 (benign) to 1 (pathogenic).

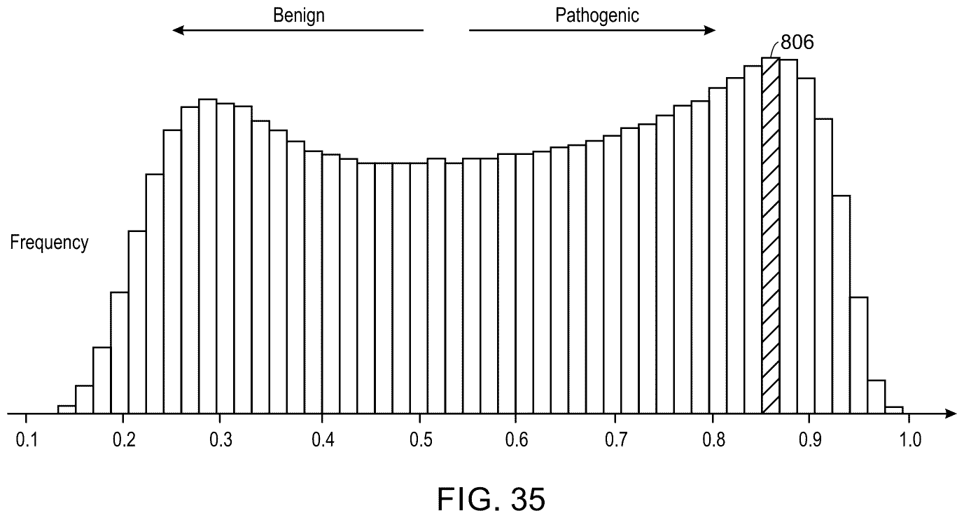

The PAI neural network model referenced herein is a deep learning network that may be used for variant pathogenicity classification (e.g., pathogenic or benign) and/or generation of a quantitative metric (e.g., a pathogenicity score) that numerically characterizes pathogenicity or the lack of pathogenicity. Outputs (e.g., pathogenicity scores and/or classifications) of a PAI model trained and used as described herein, may be used to generate additional data or diagnoses of value, such as estimation of selection effects against a range of clinically significant variants and estimation of genetic disease prevalence. Because pathogenicity scoring as described herein is based on the extent of purifying selection in humans and non-human primates, pathogenicity scores associated with pathogenic and benign variants are expected to be higher in the genes that are under strong purifying selection. On the other hand, for genes under neutral evolution or weak selection, the pathogenicity scores for pathogenic variants tend to be lower. This concept is illustrated visually in FIG. 35, where a pathogenicity score 806 for a variant is illustrated within a distribution of scores for a respective gene.

Examples and Use Cases—With the preceding in mind, it may be appreciated that genomic screening, including whole genome sequencing, may be used to derive useful fitness information for individuals or groups of individuals using techniques described herein. By way of example, whole genome sequencing on an individual or individuals may useful in determining selection burdens, lifespan projections and/or variances with respect to reference groups, quality of life projections over time, generalized health measures or characterizations, fecundity, death rates (with or without confounding or interacting variables, such as smoker status, Townsend index, health status, medical/hospitalization history), and so forth. Such information may have various real-world uses including, but not limited to, projecting healthcare needs and outcomes, projecting long-term care needs and durations, projecting investment and/or financial needs over a projected lifespan or for a period of a lifespan (e.g., retirement), estimating health care insurance needs and costs over a projected lifetime, estimating life insurance coverage needs and costs over a projected lifetime, and so forth.

By way of example, and as discussed herein, whole genome sequencing or more limited sequencing protocols may be useful in estimating or projecting risk of death within given time frames, either for a single individual or aggregated for groups of individuals. Such information may in turn be used when making financial and health related decisions for such individuals or groups of individuals.

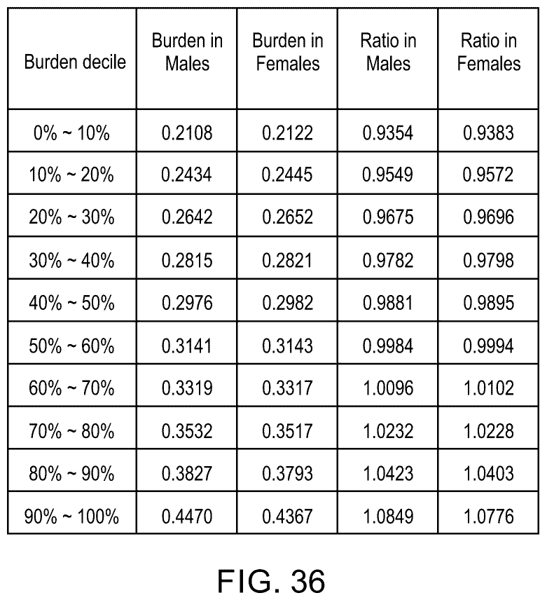

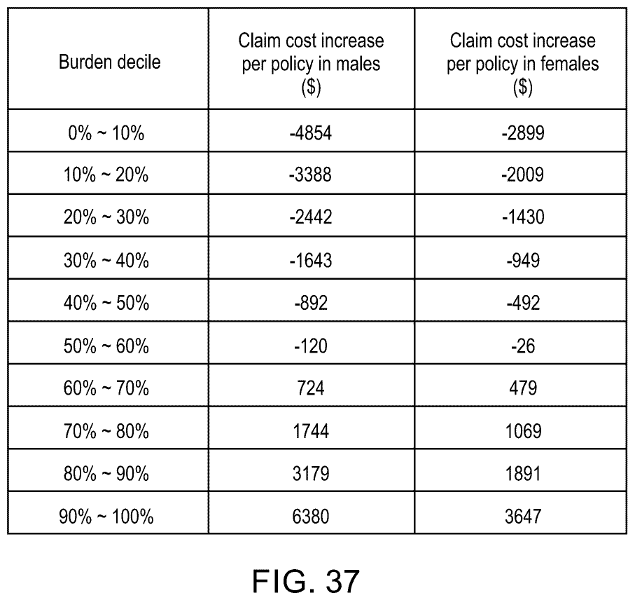

To provide real-world context, and turning to FIGS. 36 and 37, an example of an approach is described in the selection burden metrics, as described herein, may be used to characterize relative death ratios of a population (i.e., the relative increased risk of death between individuals based on selection burden). This information may, in certain embodiments be used in the determination of claim costs for life insurance policies (or, correspondingly, health insurance policies or long-term care policies).

With this in mind, and with respect to examples described below, a model population was divided into 10 groups (i.e., deciles) based on distribution of selection burden as described herein. In this context, survival curves of the ten selection burden decile groups were characterized as:

$$S ( t \vert X ) = S 0 ( t ) \hat{} exp ( log ( HR ) * ( selectionburden\_decile - selectionburden\_mean ) )$$

with a hazard ratio (HR) of 2.02 for males and of 1.93 for females. Death rates were calculated over a 20 year window (i.e., term). With this in mind, FIG. 36 illustrates, in tabular form, the death ratios by selection burden deciles of males and females compared to the overall population. In this manner, an expected death rate was calculated for males and females in the respective selection burden deciles. With refence to FIG. 36, column 1 conveys the respective selection burden deciles, columns 2 and 3 convey the average selection burdens, for males and females respectively, for each selection burden decile, and columns 4 and 5 convey the relative death rations, for males and females respectively, for each selection burden decile. As noted, such relative death ratios correspond to expected death rates within the 20 year window of interest. As may be observed as selection burden increases, relative risk of death within the 20 year window also increases.

In terms of real-world implications, such information may be useful in certain financial, medical, and long-term care contexts. By way of example, in a life insurance context such selection burden data, and corresponding relative death rates, may be useful in calculating policy pricing for individuals or groups of individuals. By way of example, and turning to FIG. 37, claim cost per policy is illustrated in tabular form for the selection burden deciles described above. In this example, claims costs per policy are observed to decrease for lower risk individuals (i.e., individuals in lower selection burden deciles). In particular, with refence to FIG. 37, column 1 conveys the respective selection burden deciles, columns 2 and 3 convey the respective claim cost differences per policy, for males and females respectively, for each selection burden decile. As may be observed, claim costs increase as the calculated selection burden of individuals increases. With this in mind, for a given individual or group of individuals, a measure of selection burden, or correspondingly derived relative death ratio or risk of death measure for a given time interval, may be used to characterize or otherwise assign individuals into respective risk categories to which different financial or health-decision conditions may be associated. Such financial or health-decision conditions may include, but are not limited to, life insurance policy premium pricing and/or term, health insurance policy premium pricing and/or term, long-term care policy premium pricing and/or term, retirement investment recommendations or options, and so forth.

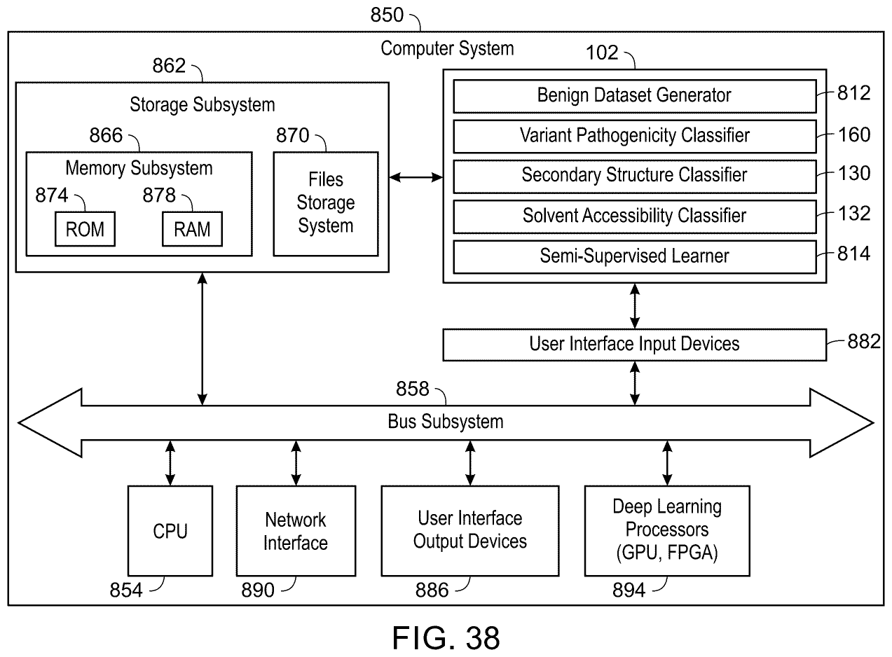

Computer System—As may be appreciated, the neural network and modeling aspects of the present discussion, as well as the analytics and processing related to fitness, lifespan, and/or fecundity estimation which may be performed using pathogenicity classifier scores), may be implemented on a computer system or systems. With this in mind, and by way of further context, FIG. 38 shows an example computing environment in which the technology presently disclosed can be operated. With respect to the actual hardware architecture that may be employed to run or support the training servers, production servers, administrative interface(s), and/or client interface(s), such hardware may be physically embodied as one or more computer systems (e.g., servers, workstations, and so forth). Examples of components which may be found in such a computer system 850 are illustrated in FIG. 38, though it should be appreciated that the present example may include components not found in all embodiments of such a system or may not illustrate all components that may be found in such a system. Further, in practice aspects of the present approach may be implemented in part or entirely in a virtual server environment or as part of a cloud platform. However, in such contexts the various virtual server instantiations will still be implemented on a hardware platform as described with respect to FIG. 38, though certain functional aspects described may be implemented at the level of the virtual server instance.

With this in mind, FIG. 38 is a simplified block diagram of a computer system 850 that can be used to implement the technology disclosed. Computer system 850 typically includes at least one processor (e.g., CPU) 854 that communicates with a number of peripheral devices via bus subsystem 858. These peripheral devices can include a storage subsystem 862 including, for example, memory devices 866 (e.g., RAM 874 and ROM 878) and a file storage subsystem 870, user interface input devices 882, user interface output devices 886, and a network interface subsystem 890. The input and output devices allow user interaction with computer system 850. Network interface subsystem 890 provides an interface to outside networks, including an interface to corresponding interface devices in other computer systems.

In one implementation in which the computer system 850 is used to implement, train, or use a pathogenicity classifier, the neural networks 102 such as benign dataset generator 812, variant pathogenicity classifier 160, secondary structure classifier 130, solvent accessibility classifier 132, and semi-supervised learner 814 are communicably linked to the storage subsystem 862 and user interface input devices 882.

In the depicted example, and in the context in which the computer system 850 is used to implement or train a neural network as discussed herein, one or more deep learning processors 894 may be present as part of the computer system 850 or otherwise in communication with the computer system 850. In such an embodiment, the deep learning processors can be GPUs or FPGAs and can be hosted by a deep learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

In the context of the computer system 850, the user interface input devices 882 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” may be construed as encompassing all possible types of devices and ways to input information into computer system 850.

User interface output devices 886 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” may be construed as encompassing all possible types of devices and ways to output information from computer system 850 to the user or to another machine or computer system.

Storage subsystem 862 stores programming and data constructs that provide the functionality of some or all of the modules and methods described herein. These software modules are generally executed by a processor 854 alone or in combination with other processors 854.

Memory 866 used in the storage subsystem 862 can include a number of memories including a main random-access memory (RAM) 878 for storage of instructions and data during program execution and a read only memory (ROM) 874 in which fixed instructions are stored. A file storage subsystem 870 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 870 in the storage subsystem 862, or in other machines accessible by the processor 854.

Bus subsystem 858 provides a mechanism for letting the various components and subsystems of computer system 850 communicate with each other as intended. Although bus subsystem 858 is shown schematically as a single bus, alternative implementations of the bus subsystem 858 can use multiple busses.

The computer system 850 itself can be of varying types including a personal computer, a portable computer, a workstation, a computer terminal, a network computer, a television, a mainframe, a stand-alone server, a server farm, a widely-distributed set of loosely networked computers, or any other data processing system or user device. Due to the ever-changing nature of computers and networks, the description of computer system 850 depicted in FIG. 38 is intended only as a specific example for purposes of illustrating the technology disclosed. Many other configurations of computer system 850 are possible having more or less components than the computer system 850 depicted in FIG. 38.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for quantifying a lifespan difference attributable to genetic mutations, the method comprising:
executing, on one or more processors of a computer system, stored instructions for quantifying a lifespan duration or effect for an individual based on a plurality of mutations, wherein the stored instructions, when executed causes the one or more processors to perform acts comprising:
receiving as an input one or more mutations of interest determined to be present in the individual;
calculating a total selection burden for the individual based on the one or more mutations of interest;
based on the total selection burden for the individual or a relative death ratio calculated based on the total selection burden, determining a cost of a financial or health product for the individual; and
providing the financial or health product to the individual at the cost.

2. The method of claim 1, wherein the financial or health product comprises one or more of a life insurance policy, a health insurance policy a long-term care policy, or a retirement investment recommendation or option.

3. The method of claim 1, further comprising:
assigning the individual to a bin or category based on the total selection burden or the relative death ratio, wherein the cost of the financial or health product for the individual is determined based upon the bin or category to which they are assigned.

4. The method of claim 1, further comprising:
automatically generating and providing one or more pharmaceutical or clinical research recommendations for the individual based on one or both of the mutations of interest for the individual or an estimated change in lifespan determined based on the total selection burden.

5. The method of claim 1, further comprising:
automatically enrolling the individual in a clinical support group or research study based on one or both of the mutations of interest for the individual or the estimated change in lifespan.

6. The method of claim 1, wherein the mutations of interest comprise one or both of missense mutations or protein truncating variants (PTVs).

7. The method of claim 1, wherein the mutations of interest are determined from a pool of mutations present in the individual based on one or both of an allele frequency for a given mutation or an estimated selection coefficient for the given mutation.

8. A method for quantifying a lifespan or lifespan difference attributable to genetic mutations, the method comprising:
executing, on one or more processors of a computer system, stored instructions for quantifying a lifespan metric for an individual based on a plurality of mutations, wherein the stored instructions, when executed causes the one or more processors to perform acts comprising:
estimating selection coefficients for a plurality of mutations determined to be present in the individual;
estimating a selection burden for the individual based upon the estimated selection coefficients;
estimating a lifespan or lifespan difference for the individual based on the selection burden for the individual;
comparing the lifespan or lifespan difference to one or more thresholds;
based upon the comparison, determining that a clinical action is indicated; and
based upon the determination that a clinical action is indicated and on the threshold comparison, determining a clinical action from among a plurality of clinical action for the individual.

9. The method of claim 8, further comprising:
displaying the clinical action or the plurality of clinical actions on a user interface of the computer system.

10. The method of claim 8, wherein the selection burden comprises a rare variant burden for the individual.

11. The method of claim 8, wherein estimating the selection burden comprises computing a multiplicative mutation burden for the individual based on the mutations of the plurality of mutations that have an allele frequency less than 0.01% and having an estimated selection coefficient greater than 0.001.

12. The method of claim 8, wherein the mutations of interest comprise one or both of missense mutations or protein truncating variants (PTVs).

13-17. (canceled)

18. A method for estimating a survival metric for an individual based on mutation data, the method comprising:
executing, on one or more processors of a computer system, stored instructions for estimating survival metrics based on a plurality of mutations, wherein the stored instructions, when executed causes the one or more processors to perform acts comprising:
accessing or acquiring gene panel data for the individual, wherein the gene panel data for the individual comprises mutation data for the individual for the genes associated with the gene panel;
calculating a selection burden for the individual based on the genes associated with the gene panel;
calculating a survival metric for the individual based on the selection burden; and
displaying on a user interface of the computer system a plurality of selectable clinical treatment options for the individual and generated based on the survival metric.

19. The method of claim 18, further comprising:
displaying on the user interface of the computer system a report comprising the survival metric for the individual based on the gene panel.

20. The method of claim 18, further comprising:
automatically enrolling the individual in a clinical support group or research study based on the survival metric.

21. The method of claim 18, wherein the gene panel comprises at least five genes per gene panel.

22. The method of claim 18, wherein selection burden comprises a multiplicative mutation burden based on mutations having an allele frequency less than 0.01% and having an estimated selection coefficient greater than 0.001.

23. The method of claim 18, wherein calculating the selection burden comprises using a multiplicative model limited to genes in the gene panel.

24. The method of claim 18, wherein calculating the survival metric comprises performing Cox proportional hazards regression using the selection burden for the individual.
